(19) United States
(12) Patent Application Publication
(10) Pub. No.: US 2024/0325066 A1
(43) Pub. Date: Oct. 3, 2024

(54) LOCAL IMPEDANCE INDICATOR IN THE TREATMENT OF CARDIAC TISSUE

(71) Applicant: CARDIOFOCUS, INC., MARLBOROUGH, MA (US)

(72) Inventors: Judy Syrkin-Nikolau, San Mateo, CA (US); Jacob I. Laughner, Medford, MA (US); David W. Hunter, Catonsville, MD (US)

(73) Assignee: CARDIOFOCUS, INC., MARLBOROUGH, MA (US)

(21) Appl. No.: 18/622,232

(22) Filed: Mar. 29, 2024

Related U.S. Application Data
(60) Provisional application No. 63/493,713, filed on Mar. 31, 2023.

(51) Int. Cl. A61B 18/00

(52) U.S. Cl. CPC A61B 18/00; A61B 2018/00267; A61B 2018/00297; A61B 2018/00577; A61B 2018/00613; A61B 2018/00642; A61B 2018/00678; A61B 2018/00732; A61B 2018/00761; A61B 2018/00767; A61B 2018/00839; A61B 2018/00875; A61B 2018/00898

(57) ABSTRACT

A treatment catheter is part of a system that includes a local impedance indicator that is displayed on a display. The local impedance indicator displays relative changes in impedance to the user in order to quickly communicate information that helps the user better understand where in the heart chamber the catheter is located.

### CROSS REFERENCE TO RELATED APPLICATION

The present application claims priority to and the benefit of U.S. patent application Ser. No. 63/493,713, filed Mar. 31, 2023, which is hereby expressly incorporated by reference in its entirety.

### BACKGROUND

Therapeutic energy can be applied to the heart and vasculature for the treatment of a variety of conditions, including atherosclerosis (particularly in the prevention of restenosis following angioplasty) and arrhythmias, such as atrial fibrillation. Atrial fibrillation is the most common sustained cardiac arrhythmia, and severely increases the risk of mortality in affected patients, particularly by causing stroke. In this phenomenon, the heart is taken out of normal sinus rhythm due to the production of erroneous electrical impulses. Atrial fibrillation is thought to be initiated in the myocardial sleeves of the pulmonary veins (PVs) due to the presence of automaticity in cells within the myocardial tissue of the PVs. Pacemaker activity from these cells is thought to result in the formation of ectopic beats that initiate atrial fibrillation. PVs are also thought to be important in the maintenance of atrial fibrillation because the chaotic architecture and electrophysiological properties of these vessels provides an environment where atrial fibrillation can be perpetuated. Thus, destruction or removal of these aberrant pacemaker cells within the myocardial sleeves of the PVs has been a goal and atrial fibrillation is often treated by delivering therapeutic energy to the pulmonary veins. However, due to reports of PV stenosis, the approach has been conventionally modified to one that targets PV antra to achieve conduction block between the PVs and the left atrium. The PV antra encompass, in addition to the pulmonary veins, the left atrial roof and posterior wall and, in the case of the right pulmonary vein antra, a portion of the interatrial septum. In some instances, this technique offers a higher success rate and a lower complication rate compared with pulmonary vein ostial isolation.

Thermal ablation therapies, especially radiofrequency (RF) ablation, are currently the “gold standard” to treat symptomatic atrial fibrillation by localized tissue necrosis. Typically, RF ablation is used to create a ring of ablation lesions around the outside of the ostium of each of the four pulmonary veins. RF current causes desiccation of tissue by creating a localized area of heat that results in discrete coagulation necrosis. The necrosed tissue acts as a conduction block thereby electrically isolating the veins.

Despite the improvements in reestablishing sinus rhythm using available methods, both success rate and safety are limited. RF ablation continues to present multiple limitations including long procedure times to perform pulmonary vein isolation with RF focal catheters, potential gaps in ablation patterns due to point-by-point ablation technique with conventional RF catheters, difficulty in creating and confirming transmural ablation lesions, char and/or gas formation at the catheter tip-tissue interface due to high temperatures, which may lead to thrombus or emboli during ablation, and thermal damage to collateral extracardiac structures, which include pulmonary vein stenosis, phrenic nerve injury, esophageal injury, atrio-esophageal fistula, peri-esophageal vagal injury, perforations, thromboembolic events, vascular complications, and acute coronary artery occlusion, to name a few. These limitations are primarily attributed to the continuous battle clinicians have faced balancing effective therapeutic dose with inappropriate energy delivery to extracardiac tissue.

Thus, while keeping the technique in clinical practice, safer and more versatile methods of removing abnormal tissue have been used, including irreversible electroporation (IRE), a non-thermal therapy based on the unrecoverable permeabilization of cell membranes caused by particular short pulses of high voltage energy. IRE has been found to be tissue-specific, triggering apoptosis rather than necrosis, and safer for the structures adjacent to the myocardium. However, thus far, the success of these IRE methodologies has been heterogeneous. In some instances, the delivery of IRE energy has resulted in incomplete block of the aberrant electrical rhythms. This may be due to a variety of factors, such as irregularity of treatment circumferentially around the pulmonary veins, lack of transmural delivery of energy or other deficiencies in the delivery of energy. In either case, atrial fibrillation is not sufficiently treated or atrial fibrillation recurs at a later time. Therefore, improvements in atrial fibrillation treatment are desired. Such treatments should be safe, effective, and lead to reduced complications. At least some of these objectives will be met by the systems, devices and methods described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

A treatment catheter is part of a system that includes a local impedance indicator that is displayed on a display. The local impedance indicator displays relative changes in impedance to the user in order to quickly communicate information that helps the user better understand where in the heart chamber the catheter is located.

In one embodiment, a system is disclosed for delivering therapeutic energy during tissue modification treatment. The system includes at least one catheter and an energy delivery body configured with the at least one catheter. The energy delivery body can be defined by a plurality of spline electrodes configured with the at least one catheter. The system further includes at least one impedance sensor configured with the at least one catheter, each of the at least one impedance sensor respectively associated with at least one of the plurality of spline electrodes.

At least one processor is configured by executing instructions stored on processor-readable media to process information associated with the at least one impedance sensor; and a display is configured to provide information processed by the at least one processor. The at least one processor is further configured to: (a) determine a baseline impedance value, wherein the baseline impedance value is based on impedance sensed by the at least one impedance sensor; (b) display, on the display, an impedance indicator that is configured with a plurality of spokes, each of the spokes respectively associated with respective ones of the spline electrodes, wherein each of the spokes is configured to represent the baseline impedance value; (c) define a threshold impedance value; (d) detect, by the at least one impedance sensor, a local impedance associated with at least one of the spline electrodes navigating via the at least one catheter about an organ; (c) determining, by the at least one processor, a change in impedance from the baseline impedance to the local impedance; (f) altering, by the at least one processor, as a function of the change in impedance, at least one respective spoke of the impedance indicator to generate an altered impedance indicator; (g) displaying, on the display, the altered impedance indicator; and wherein the tissue modification apparatus delivers the therapeutic energy via the energy delivery body.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an embodiment of a tissue modification system.

FIGS. 2A-2B illustrate embodiments of a treatment catheter configured to deliver focal therapy.

FIG. 3 illustrates a portion of the heart showing a cut-away of the right atrium and left atrium with a treatment catheter positioned therein.

FIG. 4 illustrates the repeated application of energy in point by point fashion around the left inferior pulmonary vein with the use of the treatment catheter to create a circular treatment zone.

FIG. 5 illustrates an embodiment of a waveform of a signal prescribed by an energy delivery algorithm.

FIG. 6 illustrates an example waveform prescribed by an energy delivery algorithm wherein the waveform has voltage imbalance.

FIG. 7 illustrates further examples of waveforms having unequal voltages.

FIG. 8 illustrates examples of waveforms having unequal pulse widths.

FIG. 9 illustrates an example waveform prescribed by another energy delivery algorithm wherein the waveform is monophasic, a special case of imbalance whereby there is only a positive or only a negative portion of the waveform.

FIG. 10 illustrates further examples of waveforms having monophasic pulses.

FIG. 11 illustrates examples of waveforms having phase imbalances.

FIG. 12 illustrates an example waveform prescribed by another energy delivery algorithm wherein the pulses are sinusoidal in shape rather than square.

FIG. 13 illustrates an embodiment of a treatment catheter having an energy delivery body comprised of a plurality of splines.

FIG. 14 provides a side view of the embodiment of the treatment catheter of FIG. 13.

FIG. 15 illustrates a bottom view of the treatment catheter of FIGS. 13-14.

FIG. 16 provides another perspective view of the embodiment of FIG. 13.

FIG. 17 provides a close-up illustration of a portion of the treatment catheter of FIG. 13 within the distal end of the shaft.

FIG. 18A provides an expanded illustration of elements comprising this embodiment of the treatment catheter of FIG. 13.

FIG. 18B illustrates the treatment catheter of FIG. 18A in its unexpanded state.

FIGS. 19A and 19B are views of a treatment catheter according to another embodiment.

FIGS. 20A, 20B, and 20C are views of a treatment catheter in the form of a balloon catheter according to another embodiment.

FIG. 21 illustrates a local impedance indicator at baseline.

FIG. 22 illustrates the local impedance indictor changing color, shading, and shape as the sensed local impedance values changes.

FIG. 23 is a flow chart illustrating an exemplary method for calculating and displaying a local impedance value.

FIG. 24 illustrates a display in which a directional arrow is shown that indicates the region of the energy delivery body of the treatment catheter that is in greatest contact with the tissue.

### DETAILED DESCRIPTION OF CERTAIN EMBODIMENTS

Devices, systems, and methods are provided for treating conditions of the heart, particularly the occurrence of arrhythmias, more particularly atrial fibrillation, atrial flutter, ventricular tachycardia, Wolff-Parkinson-White syndrome, and/or atrioventricular nodal reentry tachycardia, to name a few. The devices, systems and methods deliver therapeutic energy to portions of the heart to provide tissue modification, such as to the entrances to the pulmonary veins in the treatment of atrial fibrillation. Targeted specific anatomic locations include the superior vena cava, inferior vena cava, right pulmonary vein, left pulmonary vein, right atrium, right atrial appendage, left atrium, left atrial appendage, right ventricle, left ventricle, right ventricular outflow tract, left ventricular outflow tract, ventricular septum, left ventricular summit, regions of myocardial scar, myocardial infarction border zones, myocardial infarction channels, ventricular endocardium, ventricular epicardium, papillary muscles and the Purkinje system, to name a few. Treatments are delivered at isolated sites or in a connected series of treatments. Types of treatment include the creation of left atrial roof line, left atrial posterior/inferior line, posterior wall isolation, lateral mitral isthmus line, septal mitral isthmus line, left atrial appendage, right sided cavotricuspid isthmus (CTI), pulmonary vein isolation, superior vena cava isolation, vein of Marshall, lesion creation using Complex Fractionated Atrial Electrograms (CFAE), lesion creation using Focal Impulse and Rotor Modulation (FIRM), and targeted ganglia ablation. Such tissue modification creates a conduction block within the tissue to prevent the transmission of aberrant electrical signals. The devices, systems and methods are typically used in an electrophysiology lab or controlled surgical suite equipped with fluoroscopy and advanced ECG recording and monitoring capability. An electrophysiologist (EP) is typically the intended primary user of the system. The electrophysiologist will be supported by a staff of trained nurses, technicians, and potentially other electrophysiologists. Generally, the tissue modification systems include a specialized catheter, a high voltage waveform generator and at least one distinct energy delivery algorithm. Additional accessories and equipment may be utilized. Example embodiments of specialized catheter designs are provided herein and include a variety of delivery types including focal delivery, “one-shot” delivery and various possible combinations. For illustration purposes a simplified design is provided when describing the overall system. Such a simplified design provides monopolar focal therapy. However, it may be appreciated that a variety of other embodiments are also provided.

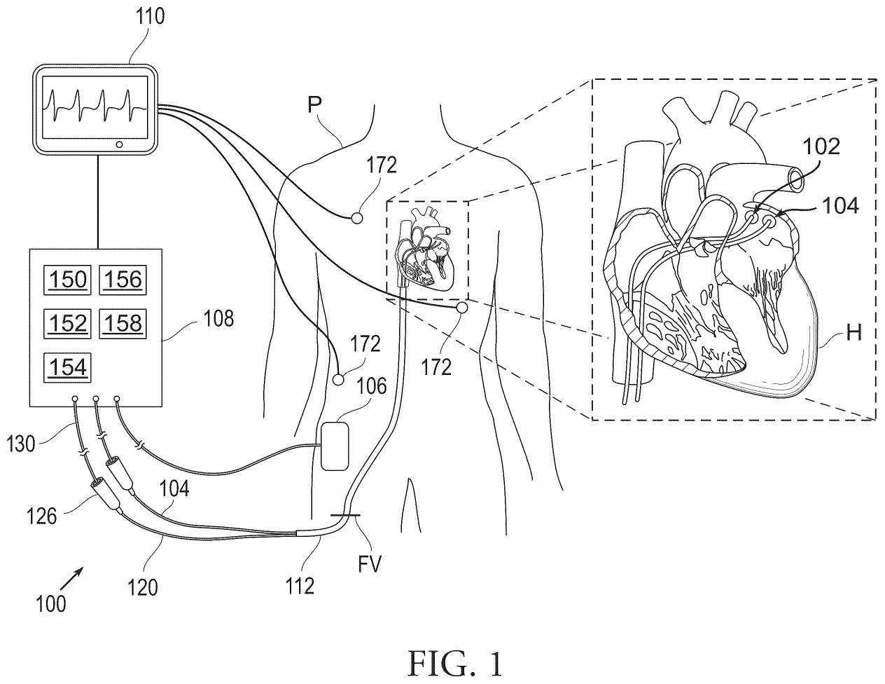

FIG. 1 illustrates an embodiment of a tissue modification system 100 comprising a treatment catheter 102, a mapping catheter 104, a return electrode 106, a waveform generator 108 and an external cardiac monitor 110. In this embodiment, the heart is accessed via the right femoral vein FV by a suitable access procedure, such as the Seldinger technique. Typically, a sheath 112 is inserted into the femoral vein FV which acts as a conduit through which various catheters and/or tools may be advanced, including the treatment catheter 102 and mapping catheter 104. It may be appreciated that in some embodiments, the treatment catheter 102 and mapping catheter 104 are combined into a single device. As illustrated in FIG. 1, the distal ends of the catheters 102, 104 are advanced through the inferior vena cava, through the right atrium, through a transseptal puncture and into the left atrium so as to access the entrances to the pulmonary veins. The mapping catheter 104 is used to perform cardiac mapping which refers to the process of identifying the temporal and spatial distributions of myocardial electrical potentials during a particular heart rhythm. Cardiac mapping during an aberrant heart rhythm aims at elucidation of the mechanisms of the heart rhythm, description of the propagation of activation from its initiation to its completion within a region of interest, and identification of the site of origin or a critical site of conduction to serve as a target for treatment. Once the desired treatment locations are identified, the treatment catheter 102 is utilized to deliver the treatment energy.

In this embodiment, the proximal end of the treatment catheter 102 is electrically connected with the waveform generator 108, wherein the generator 108 is software-controlled with regulated energy output that creates high frequency short duration energy delivered to the catheter 102. It may be appreciated that in various embodiments the output is controlled or modified to achieve a desired voltage, current, or combination thereof. In this embodiment, the proximal end of the mapping catheter 104 is also electrically connected with the waveform generator 108 and the electronics to perform the mapping procedure are included in the generator 108. However, it may be appreciated that the mapping catheter 104 may alternatively be connected with a separate external device having the capability of providing the mapping procedure, such as electroanatomic mapping (EAM) systems (e.g., CARTO® systems by Biosense Webster/Johnson & Johnson, EnSite™ systems by St. Jude Medical/Abbott, KODEX-EPD system by Philips, Rhythmia HDX™ system by Boston Scientific). Likewise, in some embodiments, a separate mapping catheter 104 is not used and the mapping features are built into the catheter 102.

In this embodiment, the generator 108 is connected with an external cardiac monitor 110 to allow coordinated delivery of energy with the cardiac signal sensed from the patient P. The generator synchronizes the energy output to the patient's cardiac rhythm. The cardiac monitor provides a trigger signal to the generator 108 when it detects the patient's cardiac cycle R-wave. This trigger signal, and the generator's algorithm, reliably synchronize the energy delivery with the patient's cardiac cycle to decrease the potential for arrhythmia due to energy delivery. Typically, a footswitch allows the user to initiate and control the delivery of the energy output. The generator user interface (UI) provides both audio and visual information to the user regarding energy delivery and the generator operating status.

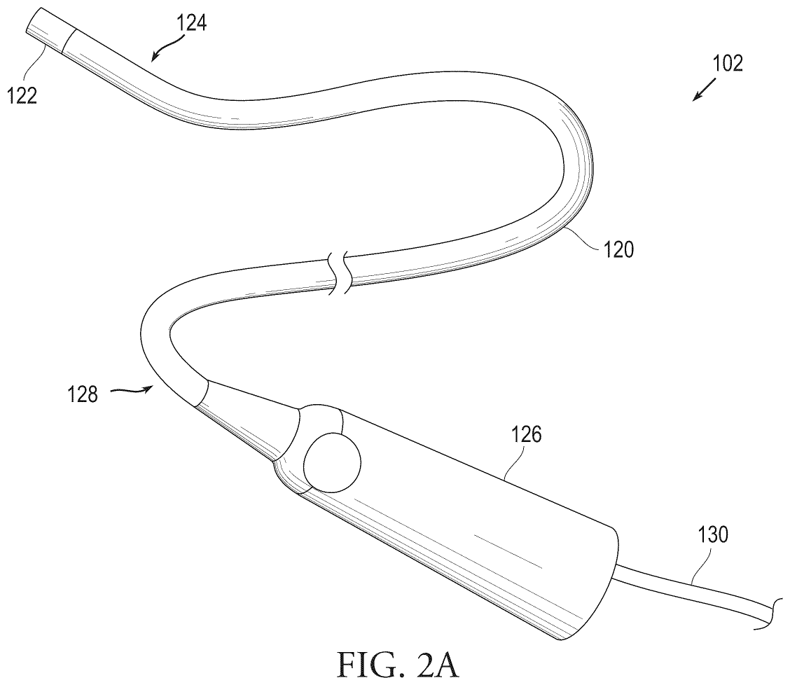

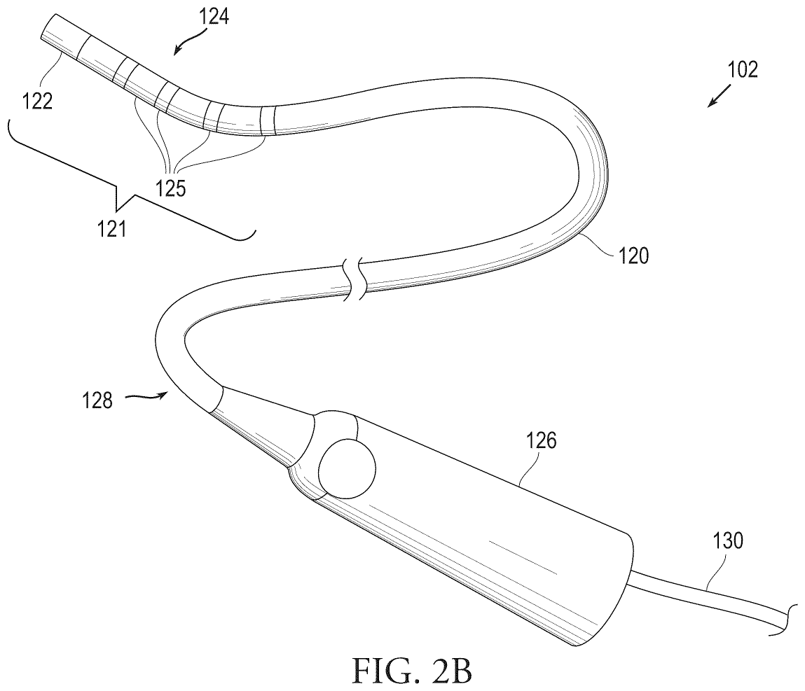

In this embodiment, the treatment catheter 102 is designed to be monopolar, wherein the distal end of the catheter 108 has as a delivery electrode 122 and the return electrode 106 is positioned upon the skin outside the body, typically on the thigh, lower back or back. FIG. 2A illustrates an embodiment of a treatment catheter 102 configured to deliver focal therapy. In this embodiment, the catheter 102 comprises an elongate shaft 120 having a delivery electrode 122 near its distal end 124 and a handle 126 near its proximal end 128. The delivery electrode 122 is shown as a “solid tip” electrode having a cylindrical shape with a distal face having a continuous surface. In some embodiments, the cylindrical shape has a diameter across its distal face of approximately 2-3 mm and a length along the shaft 120 of approximately 1 mm, 2 mm, 1-2 mm, 3 mm, 4 mm, 3-4 mm, 5 mm, 6 mm, 7 mm, 8 mm, 9 mm, 10 mm, etc. It may be appreciated that such electrodes are typically hollow yet are referred to as solid due to visual appearance. In some embodiments, the catheter 102 has an overall length of 50-150 cm, preferably 100-125 cm, more preferably 110-115 cm. Likewise, in some embodiments, it has a 7 Fr outer diameter 3-15 Fr, preferably 4-12 Fr, more preferably 7-8.5 Fr. It may be appreciated that in some embodiments, the shaft 120 has a deflectable end portion 121 and optionally the deflectable end portion 121 may have a length of 50-105 mm resulting in curves with diameters ranging from approximately 15 to 55 mm. Deflection may be achieved by a variety of mechanisms including a pull-wire which extends to the handle 126. Thus, the handle 126 is used to manipulate the catheter 102, particularly to steer the distal end 124 during delivery and treatment. Energy is provided to the catheter 102, and therefore to the delivery electrode 122, via a cable 130 that is connectable to the generator 108.

Pulsed electric fields (PEFs) are provided by the generator 108 and delivered to the tissue through the delivery electrode 122 placed on or near the targeted tissue area. It may be appreciated that in some embodiments, the delivery electrode 122 is positioned in contact with a conductive substance which is likewise in contact with the targeted tissue. Such solutions may include isotonic or hypertonic solutions. These solutions may further include adjuvant materials, such as chemotherapy or calcium, to further enhance the treatment effectiveness both for the focal treatment as well as potential regional infiltration regions of the targeted tissue types. High voltage, short duration biphasic electric pulses are then delivered through the electrode 122 in the vicinity of the target tissue. These electric pulses are provided by at least one energy delivery algorithm 152. In some embodiments, each energy delivery algorithm 152 prescribes a signal having a waveform comprising a series of energy packets wherein each energy packet comprises a series of high voltage pulses. In such embodiments, the algorithm 152 specifics parameters of the signal such as energy amplitude (e.g., voltage) and duration of applied energy, which is comprised of the number of packets, number of pulses within a packet, and the fundamental frequency of the pulse sequence, to name a few. Additional parameters may include switch time between polarities in biphasic pulses, dead time between biphasic cycles, and rest time between packets, which will be described in more detail in later sections. There may be a fixed rest period between packets, or packets may be gated to the cardiac cycle and are thus variable with the patient's heart rate. There may be a deliberate, varying rest period algorithm or no rest period may also be applied between packets. A feedback loop based on sensor information and an auto-shutoff specification, and/or the like, may be included.

Pulsed electric fields (PEFs) are provided by the generator 108 and delivered to the tissue through a delivery electrode 122 placed on or near the targeted tissue area within the heart H. The one or more energy delivery algorithms 152 specify electric signals which provide the PEF energy delivered to the cardiac tissue which cause cell death by non-thermal effects (e.g., energy below a threshold for thermal ablation; below a threshold for inducing coagulative thermal damage), reducing or avoiding inflammation, and/or preventing denaturation of stromal proteins in the anatomical structures, particularly at sensitive sites implicated in treatment morbidity or mortality, such as the phrenic nerve, esophagus, and vasculature (e.g., preventing stricture). It may be appreciated that the non-thermal energy is also not cryogenic (i.e. it is above a threshold for thermal damage caused by freezing). Thus, the temperature of the target tissue remains in a range between a baseline body temperature (such as 35° C.-37° C. but can be as low as 30° C.) and a threshold for thermal ablation. Thus, targeted ranges of tissue temperature include 30-65° C., 30-60° C., 30-55° C., 30-50° C., 30-45° C., 30-35° C. Thus, lesions in the heart tissue are not created by thermal injury as the temperature of the tissue remains below a threshold for thermal ablation (e.g., 65° C.). In addition, the impedance of the tissue typically remains below a threshold generated by thermal ablation. Charring and thermal injury of tissue changes the conductivity of the heart tissue. This increase in impedance/reduction in conductivity often indicates thermal injury and reduces the ability of the tissue to receive further energy. In some instances, the impedance of the system circuit from the cathode to the anode remains in the range of 25-250 Ω, 50-200 Ω or 75-125 Ω during delivery of PEF energy. In general, the algorithms 152 are tailored to affect tissue to a pre-determined depth and/or volume and/or to target specific types of cellular responses to the energy delivered.

It may be appreciated that in various embodiments the treatment catheter 102 includes a variety of specialized features. For example, in some embodiments, the catheter 102 includes a mechanism for real-time measurement of the contact force applied by the catheter tip to a patient's heart wall during a procedure. In some embodiments, this mechanism is included in the shaft 120 and comprises a tri-axial optical force sensor which utilizes white light interferometry. By monitoring and modifying the applied force throughout the procedure, the user is able to better control the catheter 102 so as to create more consistent and effective lesions.

In some embodiments, the catheter 102 includes one or more additional electrodes 125 (e.g., ring electrodes) positioned along the shaft 120, such as illustrated in FIG. 2B, proximal to the delivery electrode 122. In some embodiments, some or all of the additional electrodes can be used for stimulating and recording (for electrophysiological mapping), so a separate cardiac mapping catheter is not needed when using catheter 102 for lesion creation, or for other purposes such as sensing, etc.

In some embodiments, the catheter 102 includes a thermocouple temperature sensor, optionally embedded in the delivery electrode 122. Likewise, in some embodiments the catheter 102 includes a lumen which may be used for irrigation and/or suction. Typically, the lumen connects with one or more ports along the distal end of the catheter 102, such as for the injection of isotonic saline solution to irrigate or for the removal of, for example, microbubbles.

In some embodiments, the catheter 102 includes one or more sensors that can be used to determine temperature, impedance, resistance, capacitance, conductivity, permittivity, and/or conductance, to name a few. In some embodiments, one or more of the electrodes act as the one or more sensors. In other embodiments, the one or more sensors are separate from the electrodes. Sensor data can be used to plan the therapy, monitor the therapy and/or provide direct feedback via the processor 154, which can then alter the energy-delivery algorithm 152. For example, impedance measurements can be used to determine not only the initial dose to be applied but can also be used to determine the need for further treatment, or not.

Referring back to FIG. 1, in this embodiment the generator 108 includes a user interface 150, one or more energy delivery algorithms 152, a processor 154, a data storage/retrieval unit 156 (such as a memory and/or database), and an energy-storage sub-system 158 which generates and stores the energy to be delivered. In some embodiments, one or more capacitors are used for energy storage/delivery, however any other suitable energy storage element may be used. In addition, one or more communication ports are included.

In some embodiments, the generator 108 includes three sub-systems: 1) a high-energy storage system, 2) a high-voltage, medium-frequency switching amplifier, and 3) the system controller, firmware, and user interface. In this embodiment, the system controller includes a cardiac synchronization trigger monitor that allows for synchronizing the pulsed energy output to the patient's cardiac rhythm. The generator takes in alternating current (AC) mains to power multiple direct current (DC) power supplies. The generator's controller can cause the DC power supplies to charge a high-energy capacitor storage bank before energy delivery is initiated. At the initiation of therapeutic energy delivery, the generator's controller, high-energy storage banks and a bi-phasic pulse amplifier can operate simultaneously to create a high-voltage, medium frequency output.

It will be appreciated that a multitude of generator electrical architectures may be employed to execute the energy delivery algorithms. In particular, in some embodiments, advanced switching systems are used which are capable of directing the pulsed electric field circuit to the energy delivering electrodes separately from the same energy storage and high voltage delivery system. Further, generators employed in advanced energy delivery algorithms employing rapidly varying pulse parameters (e.g., voltage, frequency, etc.) or multiple energy delivery electrodes may utilize modular energy storage and/or high voltage systems, facilitating highly customizable waveform and geographical pulse delivery paradigms. It should further be appreciated that the electrical architecture described herein above is for example only, and systems delivering pulsed electric fields may or may not include additional switching amplifier components.

The user interface 150 can include a touch screen and/or more traditional buttons to allow for the operator to enter patient data, select a treatment algorithm (e.g., energy delivery algorithm 152), initiate energy delivery, view records stored on the storage/retrieval unit 156, and/or otherwise communicate with the generator 108.

In some embodiments, the user interface 150 is configured to receive operator-defined inputs. The operator-defined inputs can include a duration of energy delivery, one or more other timing aspects of the energy delivery pulse, power, and/or mode of operation, or a combination thereof. Example modes of operation can include (but are not limited to): system initiation and self-test, operator input, algorithm selection, pre-treatment system status and feedback, energy delivery, post energy delivery display or feedback, treatment data review and/or download, software update, or any combination or subcombination thereof.

As mentioned, in some embodiments the system 100 also includes a mechanism for acquiring an electrocardiogram (ECG), such as an external cardiac monitor 110, in situations wherein cardiac synchronization is desired. Example cardiac monitors are available from AccuSync Medical Research Corporation and Ivy Biomedical Systems, Inc. In some embodiments, the external cardiac monitor 110 is operatively connected to the generator 108. The cardiac monitor 110 can be used to continuously acquire an ECG signal. External electrodes 172 may be applied to the patient P to acquire the ECG. The generator 108 analyzes one or more cardiac cycles and identifies the beginning of a time period during which it is safe to apply energy to the patient P, thus providing the ability to synchronize energy delivery with the cardiac cycle. In some embodiments, this time period is within milliseconds of the R wave (of the ECG QRS complex) to avoid induction of an arrhythmia, which could occur if the energy pulse is delivered on a T wave. It will be appreciated that such cardiac synchronization is typically utilized when using monopolar energy delivery, however it may be utilized as part of other energy delivery methods.

In some embodiments, the processor 154, among other activities, modifies and/or switches between the energy-delivery algorithms, monitors the energy delivery and any sensor data, and reacts to monitored data via a feedback loop. In some embodiments, the processor 154 is configured to execute one or more algorithms for running a feedback control loop based on one or more measured system parameters (e.g., current), one or more measured tissue parameters (e.g., impedance), and/or a combination thereof.

The data storage/retrieval unit 156 stores data, such as related to the treatments delivered, and can optionally be downloaded by connecting a device (e.g., a laptop or thumb drive) to a communication port. In some embodiments, the device has local software used to direct the download of information, such as, for example, instructions stored on the data storage/retrieval unit 156 and executable by the processor 154. In some embodiments, the user interface 150 allows for the operator to select to download data to a device and/or system such as, but not limited to, a computer device, a tablet, a mobile device, a server, a workstation, a cloud computing apparatus/system, and/or the like. The communication ports, which can permit wired and/or wireless connectivity, can allow for data download, as just described but also for data upload such as uploading a custom algorithm or providing a software update.

As described herein, a variety of energy delivery algorithms 152 are programmable, or can be pre-programmed, into the generator 108, such as stored in memory or data storage/retrieval unit 156. Alternatively, energy delivery algorithms can be added into the data storage/retrieval unit to be executed by processor 154. Each of these algorithms 152 may be executed by the processor 154.

It may be appreciated that in some embodiments the system 100 includes an automated treatment delivery algorithm that dynamically responds and adjusts and/or terminates treatment in response to inputs such as temperature, impedance at various voltages or AC frequencies, treatment duration or other timing aspects of the energy delivery pulse, treatment power and/or system status.

As mentioned, in some embodiments, the cardiac monitor provides a trigger signal to the generator 108 when it detects the patient's cardiac cycle R-wave. This trigger signal, and the generator's algorithm, reliably synchronize the energy delivery with the patient's cardiac cycle to decrease the potential for arrhythmia due to energy delivery. This trigger is within milliseconds of the peak of the R wave (of the ECG QRS complex) to avoid induction of an arrhythmia, which could occur if the energy pulse is delivered on a T wave, and also to ensure that energy delivery occurs at a consistent phase of cardiac contraction. It will be appreciated that such cardiac synchronization is typically utilized when using monopolar energy delivery, however it may be utilized as part of other energy delivery methods.

In this embodiment, the generator 108 is connected with an external cardiac monitor 110 to allow coordinated delivery of energy with the cardiac signal sensed from the patient P.

In some embodiments, the generator 108 receives feedback from the cardiac monitor 110 and responds based on the received information. In some embodiments, the generator 108 receives information regarding the heart rate of the patient and either halts delivery of energy or modifies the energy delivery, such as by selecting a different energy delivery algorithm 152. In some embodiments, the generator 108 halts delivery of energy when the heart rate reaches or drops below a threshold value, such as 30 beats per minute (bpm) or 20 bpm. Optionally, the generator may provide an indicator, such as a visual or auditory indicator, when the heart rate reaches or drops below a lower threshold value, such as providing a flashing yellow light when the heart rate reaches 30 bpm and a solid red light when the heart rate reaches 20 bpm. Such safety measures ensure that the treatment energy is not delivered at an inappropriate time given that low sporadic heart rates may indicate erroneous readings.

In some embodiments, the generator 108 modifies the energy delivery based on the information from the cardiac monitor 110. For example, in some embodiments, energy delivery is provided in a 1:1 ratio when the heart rate is in a predetermined range, such as between 40 bpm and 120 bpm. This involves delivery of PEF energy at the appropriate interval of each heart beat. In some embodiments, the generator 108 modifies the energy delivery if the heart rate exceeds this range, such as if the heart rate exceeds 120 bpm. In some embodiments, the energy delivery is modified to a 2:1 ratio (two heartbeats: one delivery) wherein PEF energy is delivered at the appropriate interval of every other heart beat. It may be appreciated that various ratios of the form men (where m and n are integers) may be utilized, such as 3:1, 3:2, 4:1, 4:3 5:1, etc. It may also be appreciated that in some embodiments the heart rate may be paced to achieve a desired heart rate. Such pacing may be provided by a separate or integrated pacemaker. In some embodiments, such pacing is provided by a catheter positioned in the coronary sinus that is used for recording during procedures but is also available for pacing. Such pacing may be triggered by the generator 108 or the cardiac monitor 110.

In some embodiments, the generator 108 halts energy delivery or modifies the energy delivery based on information from other sources, such as from various sensors, including temperature sensors, impedance sensors, contact or contact force sensors, etc. In some embodiments, the generator 108 modifies energy delivery based on sensed temperature (e.g., on the catheter 102, in nearby tissue, in nearby structures, etc.). In some embodiments, energy delivery is modified to a 2:1 ratio, wherein PEF energy is delivered at the appropriate interval of every other heart beat, when the temperature reaches a predetermined threshold value. Such a modification reduces any small thermal effects, thereby reducing sensed temperature. It may be appreciated that various ratios may be utilized, such as 3:1, 3:2, 4:3, 4:1, 5:1, etc.

As mentioned previously, one or more energy delivery algorithms 152 are programmable, or can be pre-programmed, into the generator 108 for delivery to the patient P. The one or more energy delivery algorithms 152 specify electric signals which provide energy delivered to the cardiac tissue which are non-thermal (e.g., below a threshold for thermal ablation; below a threshold for inducing coagulative thermal damage), reducing or avoiding inflammation, and/or preventing denaturation of stromal proteins in the luminal structures. It may be appreciated that the non-thermal energy is also not cryogenic (i.e. it is above a threshold for thermal damage caused by freezing). Thus, the temperature of the target tissue remains in a range between a baseline body temperature (such as 35° C.-37° C. but can be as low as 30° C.) and a threshold for thermal ablation. Thus, targeted ranges of tissue temperature include 30-65° C., 30-60° C., 30-55° C., 30-50° C., 30-45° C., 30-35° C. Thus, lesions in the heart tissue are not created by thermal injury as the temperature of the tissue remains below a threshold for thermal ablation (e.g., 65° C.). In addition, the impedance of the tissue typically remains below a threshold generated by thermal ablation. Charring and thermal injury of tissue changes the conductivity of the heart tissue. This increase in impedance/reduction in conductivity often indicates thermal injury and reduces the ability of the tissue to receive further energy. In some instances, the impedance of the system circuit from the cathode to the anode remains in the range of 25-250 Ω, or 50-200 Ω during delivery of PEF energy. In general, the algorithms 152 are tailored to affect tissue to a pre-determined depth and/or volume and/or to target specific types of cellular responses to the energy delivered. However, it may be appreciated that the pulsed electric field energy described herein may be utilized more liberally than other types of energy, such as those that cause thermal injury, without negative effects. For instance, since the energy does not cause thermal injury, tissue can be over-treated to ensure sufficient lesion formation. For example, in a tissue layer that is 2 mm thick, energy sufficient to create a lesion having a depth of 6 mm can be applied to the tissue to ensure a transmural lesion. Typically, the additional energy is dissipated away from nearby critical structures through transverse tissue planes. In particular, the pericardial fluid surrounding the heart serves to dissipate energy, protecting extracardiac structures, such as the esophagus, phrenic nerve, coronary arteries, lungs, and bronchioles, from injury. This is not the case when delivering energy that creates lesions by thermal injury. In those cases, the propagation of conductive thermal energy beyond the targeted myocardial tissue can result in thermal injury to non-targeted extracardiac structures. Excessive thermal injury to the esophagus may result in esophageal ulcers that can degrade to a life-threatening atrio-esophageal fistula. Thermal injury to the phrenic nerve may result in permanent diaphragmatic paralysis leading to permanent shortness of breath and fatigue. Thermal injury to the coronary arteries can result in coronary spasm that can lead to temporary, or even permanent, chest pressure/pain. In addition, thermal lesions in the heart, in the region of the pulmonary veins can lead to pulmonary vein stenosis. Pulmonary vein stenosis is a known complication of radiofrequency ablation near the pulmonary veins in patients with atrial fibrillation. This pathologic process is related to thermal injury to the tissue that induces post-procedure fibrosis and scaring. Stenosis has been described in patients treated with many forms of thermal energy, including radiofrequency energy and cryoablation.

Since the PEF lesions described herein are not created by thermal injury, rates of “false positive” confirmation of electrical conduction blocks are also reduced. Thermal injury may result in acute myocardial edema (i.e. tissue fluid accumulation and swelling). When testing electrical conductivity across an area of thermally ablated tissue, the tissue may appear to block electrical conduction however such blocking may simply be the result of temporary edema. After a period of recovery to allow the swelling to subside, this area of treated tissue will no longer have transmural, non-conduction. In addition, acute edema due to thermal injury also diminishes the ability to re-treat an area of tissue. Once an area of tissue has undergone an amount of thermal injury, the resulting edema changes the resistive and conductive thermal properties of the tissue. Therefore, effects similar to the initial response in the tissue are difficult to obtain. Thus, any attempted re-treatment is less effective both acutely and chronically. These issues are avoided with the delivery of the energy described herein.

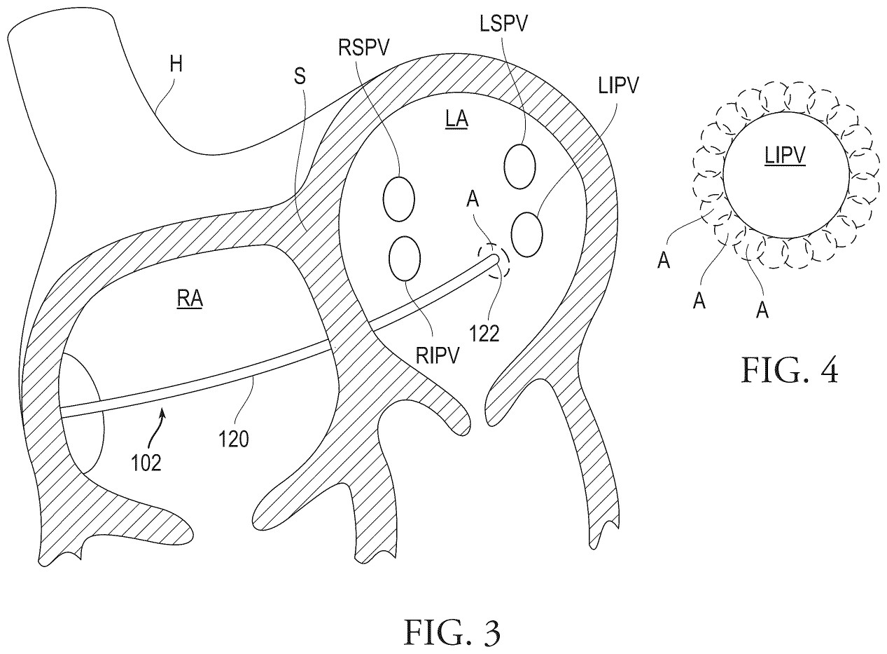

FIG. 3 illustrates a portion of the heart H showing a cut-away of the right atrium RA and left atrium LA in the treatment of atrial fibrillation. The largest pulmonary veins are the four main pulmonary veins (right superior pulmonary vein RSPV, right inferior pulmonary vein RIPV, left superior pulmonary vein LSPV and left inferior pulmonary vein LIPV), two from each lung that drain into the left atrium LA of the heart H. Each pulmonary vein is linked to a network of capillaries in the alveoli of each lung and bring oxygenated blood to the left atrium LA. The left atrial musculature extends from the left atrium LA and envelopes the proximal pulmonary veins. The superior veins, which have longer muscular sleeves, have been reported to be more arrhythmogenic than the inferior veins. In general, the length of the pulmonary vein sleeves varies between 13 mm and 25 mm. Pulmonary vein morphology has been reported to influence arrhythmogenesis. Likewise, cellular electrophysiology and other aspects of the pulmonary veins are associated with arrhythmogenesis and propagation.

A variety of methods are used to determine which tissue is targeted for treatment, such as anatomical indications and cardiac mapping. Typically, a mapping catheter is chosen to desirably fit the pulmonary vein, adapting to the size and anatomical form of the pulmonary vein. The mapping catheter allows recording of the electrograms from the ostium of the pulmonary vein and from deep within the pulmonary vein; these electrograms are displayed and timed for the user. The treatment catheter 102 is initially placed deep within the pulmonary vein and gradually withdrawn to the ostium, proximal to the mapping catheter. Mapping and treatment then commences.

The current understanding of pulmonary vein electrophysiology is that most of the fibers in the pulmonary vein are circular and do not carry conduction into the vein. The electrical conduction pathways are longitudinal fibers which extend between the left atrium LA and the pulmonary vein. Pulmonary vein isolation is achieved by ablation of these connecting longitudinal fibers. For the left-sided pulmonary veins, pacing of the distal coronary sinus tends to increase the separation of the atrial signal and the pulmonary vein potential making these more electrically visible. The signals from within the pulmonary vein are evaluated. Each individual signal consists of a far field atrial signal, which is generally of low amplitude, and a sharp local pulmonary vein spike. The earliest pulmonary vein spike represents the site of the connection of the pulmonary vein and atrium. If the pulmonary vein spike and the atrial potential are examined, on some of the poles of the mapping catheter, these electrograms are widely separated, at other sites there will be a fusion potential of the atrial and PV signal. The latter indicate the sites of the longitudinal fibers and the potential sites for treatment.

In some embodiments, the tissue surrounding the opening of the left inferior pulmonary vein LIPV is treated in a point by point fashion with the use of the treatment catheter 102 (with assistance of mapping) to create a circular treatment zone around the left inferior pulmonary vein LIPV, as illustrated in FIG. 3. In some instances, specialized navigation software can be used to allow appropriate positioning of the treatment catheter 120. The delivery electrode 122 is positioned near or against the target tissue area, and energy is provided to the delivery electrode 122 so as to create a treatment area A. Since the energy is delivered to a localized area (focal delivery), the electrical energy is concentrated over a smaller surface area, resulting in stronger effects than delivery through an electrode extending circumferentially around the lumen or ostium. It also forces the electrical energy to be delivered in a staged regional approach, mitigating the potential effect of preferential current pathways through the surrounding tissue. These preferential current pathways are regions with electrical characteristics that induce locally increased electric current flow therethrough rather than through adjacent regions. Such pathways can result in an irregular electric current distribution around the circumference of a targeted lumen, which thus can distort the electric field and cause an irregular increase in treatment effect for some regions and a lower treatment effect in other regions. This may be mitigated or avoided with the use of focal therapy which stabilizes the treatment effect around the circumference of the targeted region. Thus, by providing the energy to certain regions at a time, the electrical energy is “forced” across different regions of the circumference, ensuring an improved degree of treatment circumferential regularity. FIG. 4 illustrates the repeated application of energy in point by point fashion around the left inferior pulmonary vein LIPV with the use of the treatment catheter 102 to create a circular treatment zone. As illustrated, in this embodiment each treatment area A overlaps an adjacent treatment area A so as to create a continuous treatment zone. The size and depth of each treatment area A may depend on a variety of factors, such as parameter values, treatment times, tissue characteristics, etc. It may be appreciated that the number of treatment areas A may vary depending on a variety of factors, particularly the unique conditions of each patient's anatomy and electrophysiology. In some embodiments, the number of treatment areas A include one, two, three, four, five, six, seven, eight, nine, ten, fifteen, twenty, twenty five, thirty or more.

When all the electrical connections between the atrium and the vein have been treated, there is electrical silence within the pulmonary vein, with only the far field atrial signal being recorded. Occasionally spikes of electrical activity are seen within the pulmonary vein with no conduction to the rest of the atrium; these clearly demonstrate electrical discontinuity of the vein from the rest of the atrial myocardium.

Additional treatment areas can be created at other locations to treat arrhythmias in either the right or left atrium dependent on the clinical presentation. Testing is then performed to ensure that each targeted pulmonary vein is effectively isolated from the body of the left atrium.

### Energy Delivery Algorithms

It may be appreciated that a variety of energy delivery algorithms 152 may be used. In some embodiments, the algorithm 152 prescribes a signal having a waveform comprising a series of energy packets wherein each energy packet comprises a series of high voltage pulses. In such embodiments, the algorithm 152 specifies parameters of the signal such as energy amplitude (e.g., voltage) and duration of applied energy, which is comprised of the number of packets, number of pulses within a packet, and the fundamental frequency of the pulse sequence, to name a few. Additional parameters may include switch time between polarities in biphasic pulses, dead time between biphasic cycles, and rest time between packets, which will be described in more detail in later sections. There may be a fixed rest period between packets, or packets may be gated to the cardiac cycle and are thus variable with the patient's heart rate. There may be a deliberate, varying rest period algorithm or no rest period may also be applied between packets. A feedback loop based on sensor information and an auto-shutoff specification, and/or the like, may be included.

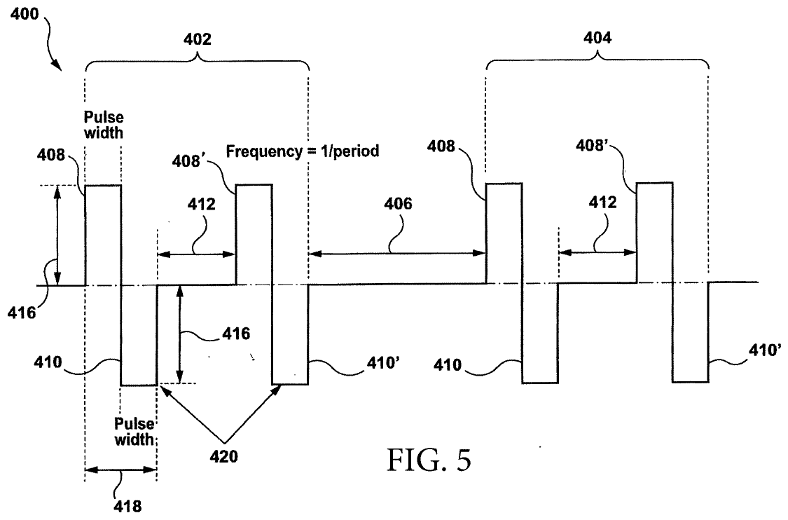

FIG. 5 illustrates an embodiment of a waveform 400 of a signal prescribed by an energy delivery algorithm 152. Here, two packets are shown, a first packet 402 and a second packet 404, wherein the packets 402, 404 are separated by a rest period 406. In this embodiment, each packet 402, 404 is comprised of a first biphasic cycle (comprising a first positive pulse peak 408 and a first negative pulse peak 410) and a second biphasic cycle (comprising a second positive pulse peak 408′ and a second negative pulse peak 410′). The first and second biphasic pulses are separated by dead time 412 (i.e. a pause) between each biphasic cycle. In this embodiment, the biphasic pulses are symmetric so that the set voltage 416 is the same for the positive and negative peaks. Here, the biphasic, symmetric waves are also square waves such that the magnitude and time of the positive voltage wave is approximately equal to the magnitude and time of the negative voltage wave.

### A. Voltage

The voltages used and considered may be the tops of square-waveforms, may be the peaks in sinusoidal or sawtooth waveforms, or may be the RMS voltage of sinusoidal or sawtooth waveforms. In some embodiments, the energy is delivered in a monopolar fashion and each high voltage pulse or the set voltage 416 is between about 500V to 10,000V, particularly about 1000V-2000V, 2000V-3000V, 3000V-3500V, 3500V-4000V, 3500V-5000V, 3500V-6000V, including all values and subranges in between including about 1000V, 2000V, 2500V, 2800V, 3000V, 3300V, 3500V, 3700V, 4000V, 4500V, 5000V, 5500V, 6000V to name a few.

It may be appreciated that the set voltage 416 may vary depending on whether the energy is delivered in a monopolar or bipolar fashion. In bipolar delivery, a lower voltage may be used due to the smaller, more directed electric field. The bipolar voltage selected for use in therapy is dependent on the separation distance of the electrodes, whereas the monopolar electrode configurations that use one or more distant dispersive pad electrodes may be delivered with less consideration for exact placement of the catheter electrode and dispersive electrode placed on the body. In monopolar electrode embodiments, larger voltages are typically used due to the dispersive behavior of the delivered energy through the body to reach the dispersive electrode, on the order of 10 cm to 100 cm effective separation distance. Conversely, in bipolar electrode configurations, the relatively close active regions of the electrodes, on the order of 0.5 mm to 10 cm, including 1 mm to 1 cm, results in a greater influence on electrical energy concentration and effective dose delivered to the tissue from the separation distance. For instance, if the targeted voltage-to-distance ratio is 3000 V/cm to evoke the desired clinical effect at the appropriate tissue depth (1.3 mm), if the separation distance is changed from 1 mm to 1.2 mm, this would result in a necessary increase in treatment voltage from 300 to about 360 V, a change of 20%.

### B. Frequency

It may be appreciated that the number of biphasic cycles per second of time is the frequency when a signal is continuous. In some embodiments, biphasic pulses are utilized to reduce undesired muscle stimulation, particularly cardiac muscle stimulation. In other embodiments, the pulse waveform is monophasic and there is no clear inherent frequency. Instead, a fundamental frequency may be considered by doubling the monophasic pulse length to derive the frequency. In some embodiments, the signal has a frequency in the range 50 kHz-1 MHZ, more particularly 50 kHz-1000 kHz. It may be appreciated that at some voltages, frequencies at or below 100-250 kHz may cause undesired muscle stimulation. Therefore, in some embodiments, the signal has a frequency in the range of 300-800 kHz, 400-800 kHz or 500-800 kHz, such as 300 kHz, 400 kHz, 450 kHz, 500 kHz, 550 kHz, 600 kHz, 650 kHz, 700 kHz, 750 kHz, 800 kHz. In addition, cardiac synchronization is typically utilized to reduce or avoid undesired cardiac muscle stimulation during sensitive rhythm periods. It may be appreciated that even higher frequencies may be used with components which minimize signal artifacts.

### C. Voltage-Frequency Balancing

The frequency of the waveform delivered may vary relative to the treatment voltage in synchrony to retain adequate treatment effect. Such synergistic changes would include the decrease in frequency, which evokes a stronger effect, combined with a decrease in voltage, which evokes a weaker effect. For instance, in some cases the treatment may be delivered using 3000 V in a monopolar fashion with a waveform frequency of 600 kHz, while in other cases the treatment may be delivered using 2000 V with a waveform frequency of 400 KHz.

### D. Packets

As mentioned, the algorithm 152 typically prescribes a signal having a waveform comprising a series of energy packets wherein each energy packet comprises a series of high voltage pulses. The cycle count 420 is half the number of pulses within each biphasic packet. Referring to FIG. 5, the first packet 402 has a cycle count 420 of two (i.e. four biphasic pulses). In some embodiments, the cycle count 420 is set between 2 and 1000 per packet, including all values and subranges in between. In some embodiments, the cycle count 420 is 5-1000 per packet, 2-10 per packet, 2-20 per packet, 2-25 per packet, 10-20 per packet, 20 per packet, 20-30 per packet, 25 per packet, 20-40 per packet, 30 per packet, 20-50 per packet, 30-60 per packet, up to 60 per packet, up to 80 per packet, up to 100 per packet, up to 1,000 per packet or up to 2,000 per packet, including all values and subranges in between.

The packet duration is determined by the cycle count, among other factors. For a matching pulse duration (or sequence of positive and negative pulse durations for biphasic waveforms), the higher the cycle count, the longer the packet duration and the larger the quantity of energy delivered. In some embodiments, packet durations are in the range of approximately 50 to 1000 microseconds, such as 50 μs, 60 μs, 70 μs, 80 μs, 90 μs, 100 μs, 125 μs, 150 μs, 175 μs, 200 μs, 250 μs, 100 to 250 μs, 150 to 250 μs, 200 to 250 μs, 500 to 1000 μs to name a few. In other embodiments, the packet durations are in the range of approximately 100 to 1000 microseconds, such as 150 μs, 200 μs, 250 μs, 500 μs, or 1000 μs.

The number of packets delivered during treatment, or packet count, typically includes 1 to 250 packets including all values and subranges in between. In some embodiments, the number of packets delivered during treatment comprises 10 packets, 15 packets, 20 packets, 25 packets, 30 packets or greater than 30 packets.

### E. Rest Period

In some embodiments, the time between packets, referred to as the rest period 406, is set between about 0.001 seconds and about 5 seconds, including all values and subranges in between. In other embodiments, the rest period 406 ranges from about 0.01-0.1 seconds, including all values and subranges in between. In some embodiments, the rest period 406 is approximately 0.5 ms-500 ms, 1-250 ms, or 10-100 ms to name a few.

### F. Batches

In some embodiments, the signal is synced with the cardiac rhythm so that each packet is delivered synchronously within a designated period relative to the heartbeats, thus the rest periods coincide with the heartbeats. It may be appreciated that the packets that are delivered within each designated period relative to the heartbeats may be considered a batch or bundle. Thus, each batch has a desired number of packets so that at the end of a treatment period, the total desired number of packets have been delivered. Each batch may have the same number of packets, however in some embodiments, batches have varying numbers of packets.

In some embodiments, only one packet is delivered between heartbeats. In such instances, the rest period may be considered the same as the period between batches. However, when more than one packet is delivered between batches, the rest time is typically different than the period between batches. In such instances, the rest time is typically much smaller than the period between batches. In some embodiments, each batch includes 1-10 packets, 1-5 packets, 1-4 packets, 1-3 packets, 2-3 packets, 2 packets, 3 packets, 4 packets 5 packets, 5-10 packets, to name a few. In some embodiments, each batch has a period of 0.5 ms-1sec, 1 ms-1 sec, 10 ms-1 sec, 10 ms-100 ms, to name a few. In some embodiments, the period between batches is variable, depending on the heart rate of the patient. In some instances, the period between batches is 0.25-5 seconds.

Treatment of a tissue area ensues until a desired number of batches are delivered to the tissue area. In some embodiments, 2-50 batches are delivered per treatment, wherein a treatment is considered treatment of a particular tissue area. In other embodiments, treatments include 5-40 batches, 5-30 batches, 5-20 batches, 5-10 batches, 5 batches, 6 batches, 7 batches, 8 batches, 9 batches, 10 batches, 10-15 batches, etc.

### G. Switch Time and Dead Time

A switch time is a delay or period of no energy that is delivered between the positive and negative peaks of a biphasic pulse, as illustrated in FIG. 5. In some embodiments, the switch time ranges between about 0 to about 1 microsecond, including all values and subranges in between. In other embodiments, the switch time ranges between 1 and 20 microseconds, including all values and subranges in between. In other embodiments, the switch time ranges between about 2 to about 8 microsecond, including all values and subranges in between.

Delays may also be interjected between each biphasic cycle, referred to as “dead-time”. Dead time occurs within a packet, but between biphasic pulses. This is in contrast to rest periods which occur between packets. In other embodiments, the dead time 412 is in a range of approximately 0 to 0.5 microseconds, 0 to 10 microseconds, 2 to 5 microseconds, 0 to 20 microseconds, about 0 to about 100 microseconds, or about 0 to about 100 milliseconds, including all values and subranges in between. In some embodiments, the dead time 412 is in the range of 0.2 to 0.3 microseconds. Dead time may also be used to define a period between separate, monophasic, pulses within a packet.

Delays, such as switch times and dead times, are introduced to a packet to reduce the effects of biphasic cancellation within the waveform. In some instances, the switch time and dead time are both increased together to strengthen the effect. In other instances, only switch time or only dead time are increased to induce this effect.

### G. Waveforms

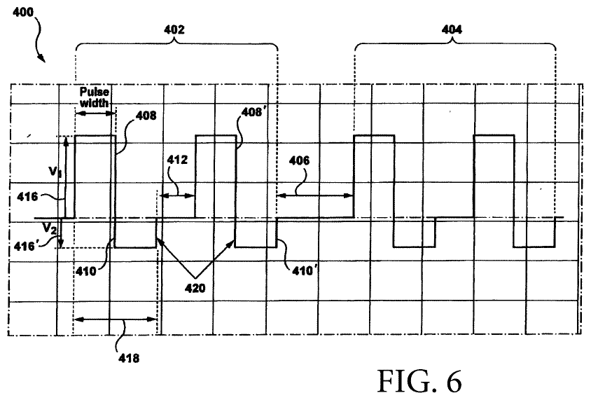

FIG. 5 illustrated an embodiment of a waveform 400 having symmetric pulses such that the voltage and duration of pulse in one direction (i.e., positive or negative) is equal to the voltage and duration of pulse in the other direction. FIG. 6 illustrates an example waveform 400 prescribed by another energy delivery algorithm 152 wherein the waveform 400 has voltage imbalance. Here, two packets are shown, a first packet 402 and a second packet 404, wherein the packets 402, 404 are separated by a rest period 406. In this embodiment, each packet 402, 404 is comprised of a first biphasic cycle (comprising a first positive pulse peak 408 having a first voltage V1 and a first negative pulse peak 410 having a second voltage V2) and a second biphasic cycle (comprising a second positive pulse peak 408′ having first voltage V1 and a second negative pulse peak 410′ having a second voltage V2). Here the first voltage V1 is greater than the second voltage V2. The first and second biphasic cycles are separated by dead time 412 between each pulse. Thus, the voltage in one direction (i.e., positive or negative) is greater than the voltage in the other direction so that the area under the positive portion of the curve does not equal the area under the negative portion of the curve. This unbalanced waveform may result in a more pronounced treatment effect as the dominant positive or negative amplitude leads to a longer duration of same charge cell membrane charge potential. In this embodiment, the first positive peak 408 has a set voltage 416 (V1) that is larger than the set voltage 416′ (V2) of the first negative peak 410. FIG. 7 illustrates further examples of waveforms having unequal voltages. Here, four different types of packets are shown in a single diagram for condensed illustration. The first packet 402 is comprised of pulses having unequal voltages but equal pulse widths, along with no switch times and dead times. Thus, the first packet 402 is comprised of four biphasic pulses, each comprising a positive peak 408 having a first voltage V1 and a negative peak 410 having a second voltage V2). Here the first voltage V1 is greater than the second voltage V2. The second packet 404 is comprised of pulses having unequal voltages but symmetric pulse widths (as in the first pulse 402), with switch times equal to dead times. The third packet 405 is comprised of pulses having unequal voltages but symmetric pulse widths (as in the first pulse 402), with switch times that are shorter than dead times. The fourth packet 407 is comprised of pulses having unequal voltages but symmetric pulse widths (as in the first pulse 402), with switch times that are greater than dead times. It may be appreciated that in some embodiments, the positive and negative phases of biphasic waveform are not identical, but are balanced, where the voltage in one direction (i.e., positive or negative), is greater than the voltage in the other direction but the length of the pulse is calculated such that the area under the curve of the positive phase equals the area under the curve of the negative phase.

In some embodiments, imbalance includes pulses having pulse widths of unequal duration. In some embodiments, the biphasic waveform is unbalanced, such that the voltage in one direction is equal to the voltage in the other direction, but the duration of one direction (i.e., positive or negative) is greater than the duration of the other direction, so that the area under the curve of the positive portion of the waveform does not equal the area under the negative portion of the waveform.

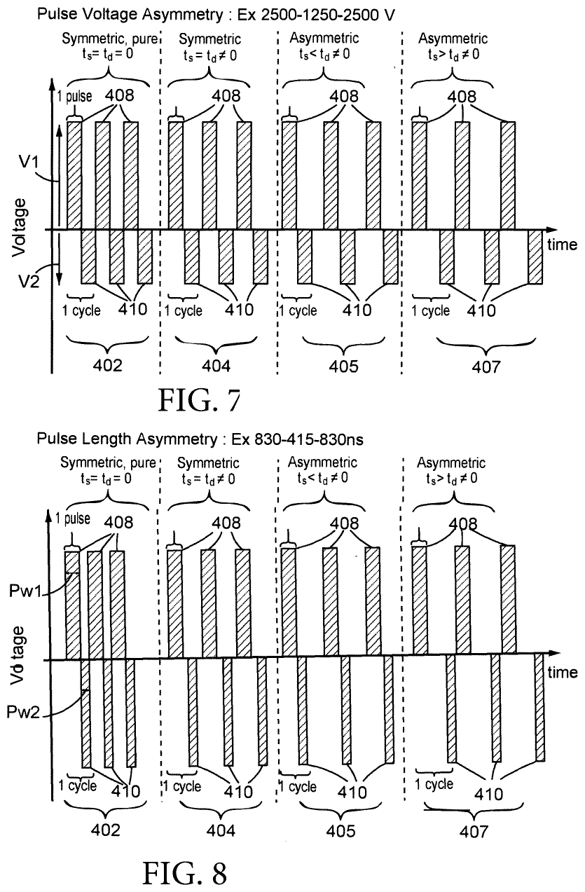

FIG. 8 illustrates further examples of waveforms having unequal pulse widths. Here, four different types of packets are shown in a single diagram for condensed illustration. The first packet 402 is comprised of pulses having equal voltages but unequal pulse widths, along with no switch times and dead times. Thus, the first packet 402 is comprised of four biphasic pulses, each comprising a positive peak 408 having a first pulse width PW1 and a negative peak 410 having a second pulse width PW2). Here the first pulse width PW1 is greater than the second pulse width PW2. The second packet 404 is comprised of pulses having equal voltages but unequal pulse widths (as in the first pulse 402), with switch times equal to dead times. The third packet 405 is comprised of pulses having equal voltages but unequal pulse widths (as in the first pulse 402), with switch times that are shorter than dead times. The fourth packet 407 is comprised of pulses having equal voltages but unequal pulse widths (as in the first pulse 402), with switch times that are greater than dead times.

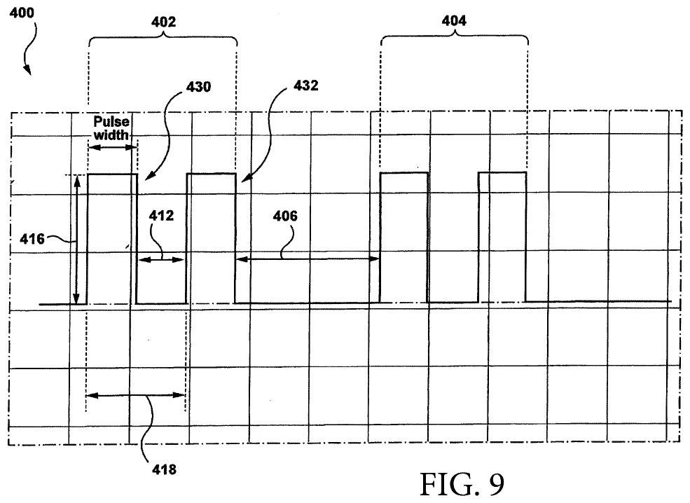

FIG. 9 illustrates an example waveform 400 prescribed by another energy delivery algorithm 152 wherein the waveform is monophasic, a special case of imbalance whereby there is only a positive or only a negative portion of the waveform. Here, two packets are shown, a first packet 402 and a second packet 404, wherein the packets 402, 404 are separated by a rest period 406. In this embodiment, each packet 402, 404 is comprised of a first monophasic pulse 430 and a second monophasic pulse 432. The first and second monophasic pulses 430, 432 are separated by dead time 412 between each pulse. This monophasic waveform could lead to a more desirable treatment effect as the same charge cell membrane potential is maintained for longer durations. However, adjacent muscle groups will be more stimulated by the monophasic waveform, compared to a biphasic waveform.

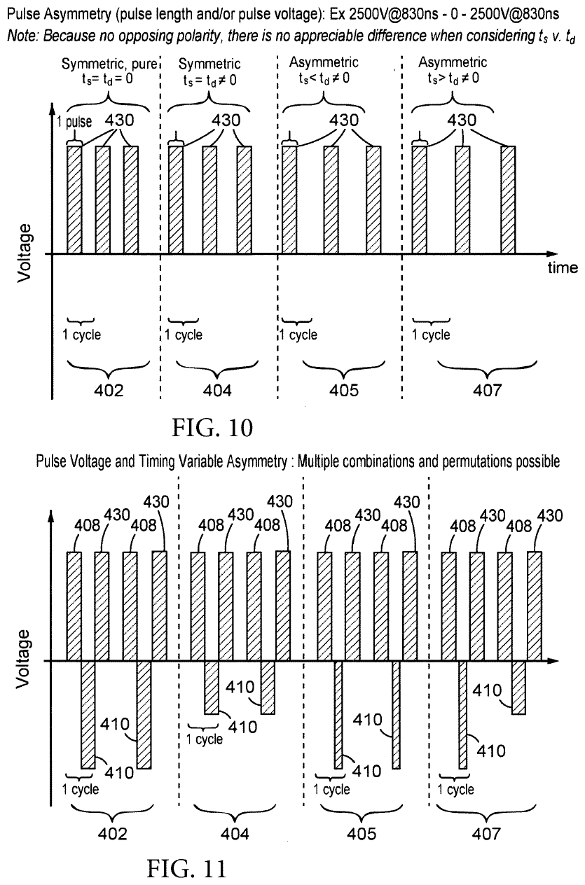

FIG. 10 illustrates further examples of waveforms having monophasic pulses. Here, four different types of packets are shown in a single diagram for condensed illustration. The first packet 402 is comprised of pulses having identical voltages and pulse widths, with no switch times (because the pulses are monophasic) and a dead time equal to the active time. In some cases, there may be less dead time duration than the active time of a given pulse. Thus, the first packet 402 is comprised of three monophasic pulses 430, each comprising a positive peak. In instances where the dead time is equal to the active time, the waveform may be considered unbalanced with a fundamental frequency representing a cycle period of 2×the active time and no dead time. The second packet 404 is comprised of monophasic pulses 430 having equal voltages and pulse widths (as in the first packet 402), with larger dead times. The third packet 405 is comprised of monophasic pulses 430 having equal voltages and pulse widths (as in the first packet 402), and even larger dead times. The fourth packet 407 is comprised of monophasic pulses 430 having equal voltages and pulse widths (as in the first packet 402), with yet larger dead times.

In some embodiments, an unbalanced waveform is achieved by delivering more than one pulse in one polarity before reversing to an unequal number of pulses in the opposite polarity. FIG. 11 illustrates further examples of waveforms having such phase imbalances. Here, four different types of packets are shown in a single diagram for condensed illustration. The first packet 402 is comprised of four cycles having equal voltages and pulse widths, however, opposite polarity pulses are intermixed with monophasic pulses. Thus, the first cycle comprises a positive peak 408 and a negative peak 410. The second cycle is monophasic, comprising a single positive pulse with no subsequent negative pulse 430. This then repeats. The second packet 404 is comprised of intermixed biphasic and monophasic cycles (as in the first packet 402), however the pulses have unequal voltages. The third packet 405 is comprised of intermixed biphasic and monophasic cycles (as in the first packet 402), however the pulses have unequal pulse widths. The fourth packet 407 is comprised of intermixed biphasic and monophasic pulses (as in the first packet 402), however the pulses have unequal voltages and unequal pulse widths. Thus, multiple combinations and permutations are possible.

### H. Waveform Shapes

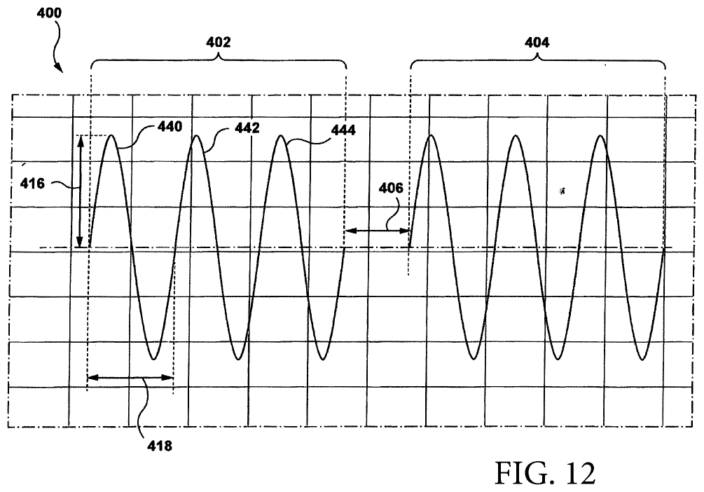

FIG. 12 illustrates an example waveform 400 prescribed by another energy delivery algorithm 152 wherein the pulses are sinusoidal in shape rather than square. Again, two packets are shown, a first packet 402 and a second packet 404, wherein the packets 402, 404 are separated by a rest period 406. In this embodiment, each packet 402, 404 is comprised of three biphasic pulses 440, 442, 444. And, rather than square waves, these pulses 440, 442, 444 are sinusoidal in shape. One benefit of a sinusoidal shape is that it is balanced or symmetrical, whereby each phase is equal in shape. Balancing may assist in reducing undesired muscle stimulation. It may be appreciated that in other embodiments the pulses have decay-shaped waveforms.

Energy delivery may be actuated by a variety of mechanisms, such as with the use of a button on the catheter 102 or a foot switch operatively connected to the generator 104. Such actuation typically provides a single energy dose. The energy dose is defined by the number of packets delivered and the voltage of the packets. Each energy dose delivered to the tissue maintains the temperature at or in the tissue below a threshold for thermal ablation. In addition, the doses may be titrated or moderated over time so as to further reduce or eliminate thermal build up during the treatment procedure. Instead of inducing thermal damage, defined as protein coagulation at sites of danger to therapy, the energy dose provide energy at a level which induces treats the condition without damaging sensitive tissues.

Another embodiment includes the selection of energy delivery based on the determination of factors that determine optimal delivery through a particular electrode. The energy dose can be scaled or otherwise broken down to deliver an equivalent amount of energy based on which electrodes meet the criteria for optimal delivery.

### Treatment Catheter Designs

The systems and devices described herein may be used with a variety of types and styles of treatment catheters 102. In some embodiments, the treatment catheters 102 are designed to deliver focal therapy and in other embodiments, the treatment catheters 102 are designed to deliver “one shot” therapy. Focal therapy is considered to be a therapy wherein the energy is delivered in a sequence, such as the repeated application of energy in point by point fashion, such as around a pulmonary vein to create a circular treatment zone, such as previously illustrated in FIG. 4, or along a line, curve, etc. One shot therapy is considered to be a therapy wherein the energy is delivered via an energy delivery body or delivery electrode(s) to the entire circumference of the entrance to the pulmonary vein in “one shot”, however such delivery may be repeated if desired. This may optionally include rotation of the energy delivery body or electrode 122 between “shots” if desired.

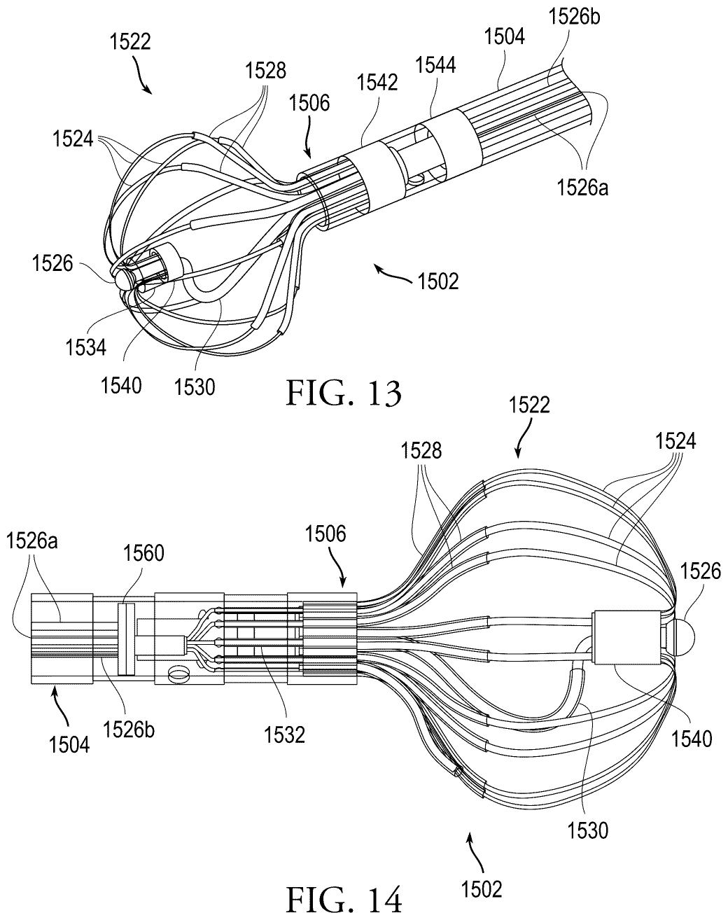

FIGS. 13-17 and FIGS. 18A-18B illustrate an embodiment of a treatment catheter 1502 configured for focal delivery. Here, the treatment catheter 1502 comprises a shaft 1504 having a distal end 1506 (FIG. 14) and an energy delivery body 1522 disposed near the distal end 1506. Here, the energy delivery body 1522 comprises a plurality of conductive splines 1524 forming a convex distal face. In addition, in this embodiment, the energy delivery body 1522 includes a distal tip electrode 1526. Here, the distal tip electrode 1526 is disposed along the center of the convex distal face. This provides additional energy delivery to the tissue upon which the convex distal face is positioned. This assists in avoiding any potential low or missing area of energy delivery, such as would generate a donut shaped lesion in the tissue. Thus, a continuous, circular lesion is created.

It may be appreciated that each spline 1524 may act as an electrode wherein the splines are energized in unison, independently or in groups. Thus, in some embodiments, the energy is delivered from all or a subset of the plurality of splines 1524 in unison so that the energy delivery body 1522 delivers energy in a monopolar fashion with the use of at least one remote return electrode. Likewise, the distal tip electrode 1526 may additionally be energized in unison with the splines 1524 so as to deliver energy is unison in a monopolar fashion. In other embodiments, the energy is delivered between selected splines or selected groups of splines so that the energy is delivered in a bipolar fashion. Likewise, combinations of one or more splines 1524 and the distal tip electrode 1526 may be energized to deliver energy in a bipolar fashion. Further, energy may be delivered from the tip electrode 1526 alone, without energy delivery from one or more splines 1524, and vice versa wherein energy is delivered from one or more splines 1524 and not from the tip electrode 1526. It may be appreciated that the splines 1524 may be wires, flat wires, struts, planks, strips or the like. In this embodiment, the splines 1524 are comprised of shape memory material, such as nitinol flat wire. In this embodiment, the nitinol flat wire has a platinum core for improved visualization under fluoroscopy. In this embodiment, the plurality of splines 1524 are partially covered by insulative material 1528. Here, the insulative material 1528 is located on the proximal side of the energy delivery body 1522 so that energy conducted to the plurality of splines 1524 resists delivery through the insulative material 1528, directing the energy to the uninsulated portion of the plurality of splines 1524 facing distally. Consequently, the energy provided to the energy delivery body 1522 is focused in the distal direction. Since the distal convex face is positionable against the target tissue area, the energy is efficiently directed toward the target tissue area without loss of energy out the proximal side of the energy delivery body 1522. This conserves energy and reduces energy sink to surrounding blood, etc.

In this embodiment, the distal tip electrode 1526 is comprised of platinum-iridium and has a ball shape. It may be appreciated that other suitable materials may be used and other shapes may be used, such as a flat shape, oblong shape or a pointed shape, to name a few. In some embodiments, the distal tip electrode 1526 facilitates directing the treatment catheter 1502 to the target tissue area. This is achieved by using the distal tip electrode 1526 to detect areas of active cardiac tissue that are still needing to be treated. Thus, by reading the electrogram, the next placement of the catheter can be determined. In some embodiments, the distal tip electrode 1526 is used for recording data as will be described in later sections.

The energy delivery body 1522 is transitionable between a collapsed configuration and an expanded configuration. FIG. 13 illustrates the energy delivery body 1522 in an expanded configuration. To collapse the energy delivery body 1522, a sheath or delivery tube is advanceable over the shaft 1504 in the distal direction. As the sheath is advanced over the energy delivery body 1522, the flexibility of the splines 1524 allow the splines 1524 to straighten, thereby causing the profile of the energy delivery body 1522 to flatten and fit within the sheath. In addition, it may be appreciated that such straightening of the splines 1524 lengthens the distance between the distal tip electrode 1526 and the distal end 1506 of the shaft 1504. For this reason, a tip electrode wire 1530 that conducts energy through the shaft 1504 to the distal tip electrode 1526 is slacked when the energy delivery body 1522 is in the expanded configuration as shown in FIG. 13. Such slack allows for lengthening when in the collapsed configuration.

It may be appreciated that the catheter 1504 is delivered to the target treatment area within the body while the energy delivery body 1522 is collapsed and held within a sheath, sleeve or delivery device. Upon desired positioning in the body, the energy delivery body 1522 is then advanced from the sheath (or the sheath is retracted) so that the energy delivery body 1522 is exposed. Such exposure allows the energy delivery body 1522 to self-expand to the expanded configuration. It may be appreciated that in other embodiments, the energy delivery body 1522 may be expanded by other mechanism, such as by retraction of a plunger connected with the distal tip electrode 1526 or by expansion of a flexible expandable member (e.g., a balloon) within the energy delivery body 1522. However, the embodiment of FIG. 13 provides an energy delivery body 1522 that is free of a central shaft creating a hollow rounded cage. This allows for additional flexibility of the energy delivery body 1522. For example, when pressing the convex distal face against target tissue, additional force may allow the plurality of splines 1524 to flex outwardly, increasing the diameter of the convex distal face. Likewise, movement of the shaft 1504 while keeping the convex distal face stationary, may allow increased force against the tissue in the direction of movement due to flexing of the splines 1524. For example, movement of the shaft 1504 to the right during engagement may increase engagement of the splines 1524 on the right side of the convex distal face and allow more force against this area of the tissue. In addition, such increased flexibility may also allow the energy delivery body 1522 to be steered more easily, such as to bend more freely with the use of pullwires or the like.

FIG. 14 provides a side view of the embodiment of the treatment catheter 1502 of FIG. 13. Again, the plurality of splines 1524 are illustrated in an expanded configuration wherein the energy delivery body 1522 forms a ball-shaped cage having a convex distal face. In this embodiment, the plurality of splines 1524 come together, spaced around a shaft plug 1532 within the shaft 1504, in an evenly spaced circumferential array. In this embodiment, each of the splines 1524 are connected to a conductive wire that extends through the shaft 1504 for connection with the energy generator. In this embodiment, the plurality of splines 1524 terminate around the distal tip electrode 1526 by curving and bending inward for mounting on a tip inner 1534, as can be seen by referring back to FIG. 13. Thus, in this embodiment, the splines 1524 are evenly spaced in a circumferential array around the tip inner 1534. By curving and bending inward around the distal tip electrode 1526, a smooth, distal face is formed for positioning against the target tissue.

In this embodiment, the energy delivery body 1522 includes a sensing electrode 1540 positioned within the energy delivery body 1522 so as to avoid contact with the target tissue. In this embodiment, the sensing electrode 1540 is disposed proximally of the distal tip electrode 1526 (i.e. behind the distal tip electrode 1526) within the rounded cage of the plurality of splines 1524. Here, the sensing electrode 1540 comprises a ring electrode, such as a single 0.030″ ring electrode, extending around the tip inner 1534. In this embodiment, additional electrodes 1542, 1544 are disposed along the shaft 1504, such as two 0.070″ ring electrodes, proximal to the energy delivery body 1522. In this embodiment, the electrodes 1540, 1542, 1544 are comprised of platinum-iridium and also serve as marker bands for visualization under fluroscopy.

The sensing electrode 1540 and the additional electrodes 1542, 1544 are typically used for sensing ECG signals and also for providing information to an electroanatomic mapping system. For example, when sensing ECG signals, a user can verify or confirm location of the treatment catheter 1502 in the heart based on the sensed ECG signals. When approaching a ventricle, a user may verify such approach by checking that ventricular signals increase in amplitude. Likewise, in some instances, impedance measurements are tracked by the sensing electrode 1540 and/or the additional electrodes 1542, 1544. Electroanatomic mapping systems use such impedance measurements to visualize the location of these electrodes 1540, 1542, 1544, and therefore the location of the treatment catheter 1502, within the heart.

Referring to FIG. 14, this embodiment also includes a steering mechanism. In this embodiment, the steering mechanism comprises a pullring 1560 disposed along the shaft 1504. The pullring 1560 is connected with one or more pullwires. In this embodiment, the pullring 1560 is connected to a first pullwire 1562a and a second pullwire 1562b, wherein the pullwires 1562a, 1562b are attached to the pullring 1560 on opposite sides. The pullwires 1562a, 1562b extend toward the proximal end of the shaft 1504 so that the distal end 1506 can be manipulated remotely. Pulling of the first pullwire 1562a causes the distal end 1506 and therefore energy delivery body 1522 to bend in the direction of the first pullwire 1562a (e.g., to the left) and pulling of the second pullwire 1562b causes the distal end 1506 and therefore energy delivery body 1522 to bend in the direction of the secondpullwire 1562b (e.g., to the right). It may be appreciated that any suitable number of pullwires may be present to steer in a variety of directions. Likewise, other steering mechanisms may be used instead of or addition to the steering mechanisms described herein.

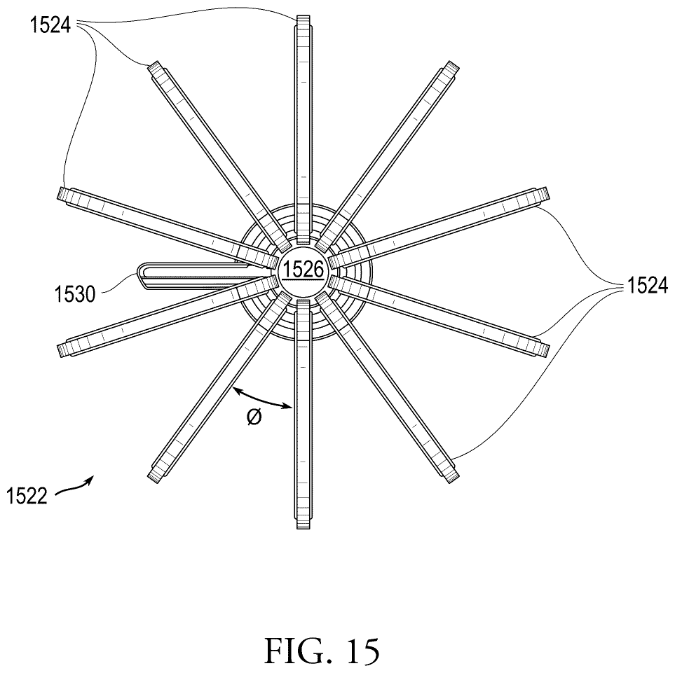

FIG. 15 illustrates a bottom view of the treatment catheter 1502 of FIGS. 13-14, facing the convex distal face of the energy delivery body 1522. As illustrated, in this embodiment, the energy delivery body 1522 includes ten splines 1524, however any suitable number of splines 1524 may be present including one, two, three, four, five, six, seven, eight, nine, ten, eleven, twelve or more. Here, a distal portion of each spline 1524 extends radially outwardly from the distal tip electrode 1526 before curving back toward the proximal direction to form a ball, sphere or rounded cage shape. Thus, each of these spline surfaces are uninsulated and deliver energy to the tissue. In this embodiment, an angle θ forms between each spline 1524, therefore each angle θ is 36 degrees. It may be appreciated that the angle θ will vary depending on the number of splines 1524, however such angle θ are typically in a range of 10-45 degrees, such as 10-20 degrees, 20-30 degrees or 30-45 degrees. With fewer splines 1524, such angle θ may increase to 50 degrees, 60 degrees, 70 degrees, 80 degrees or 90 degrees, to name a few.

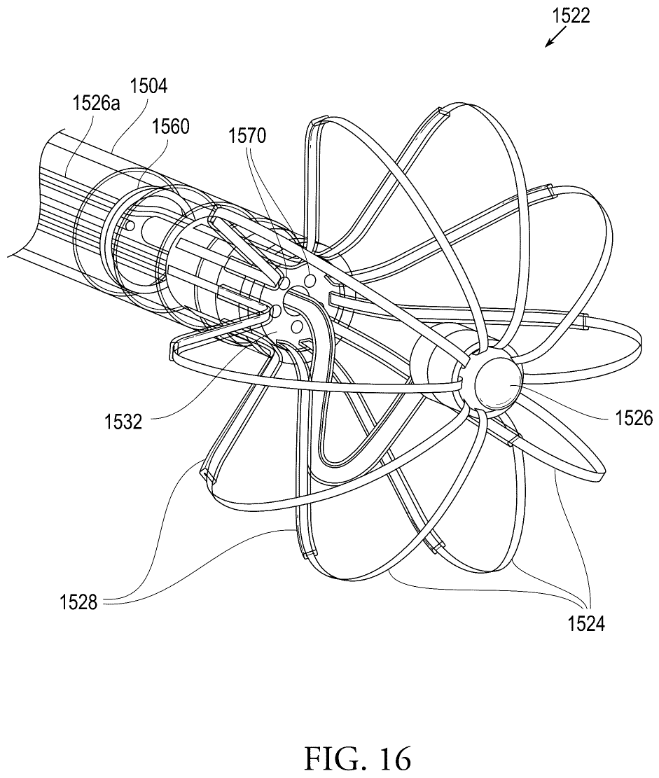

FIG. 16 provides another perspective view of the embodiment of FIG. 13. In this view, irrigation ports 1570 are visible along the distal face of the shaft plug 1532. The irrigation ports 1570 are so positioned so as to deliver irrigation fluid to a proximal end of the energy delivery body 1522 allowing flow toward a distal end of the energy delivery body 1522 (i.e. toward the tip inner 1534 and distal tip electrode 1526). Such irrigation assists in reducing any potential blood clot formation along the energy delivery body 1522. In some instances, blood clotting may be more likely between elements such as splines 1524 which are closely spaced and are positioned in a blood-filled field. As illustrated in FIG. 16, the distance between the splines 1524 tapers toward the proximal end of the energy delivery body 1522 and toward the distal end of the energy delivery body 1522. Such areas are more prone to blood clotting due to increased blood stagnation in these areas. Stagnant blood may clot causing risks to the patient. Using a flow of irrigation fluid, such as saline, in and around the splines 1524 decreases the likelihood of clots forming.

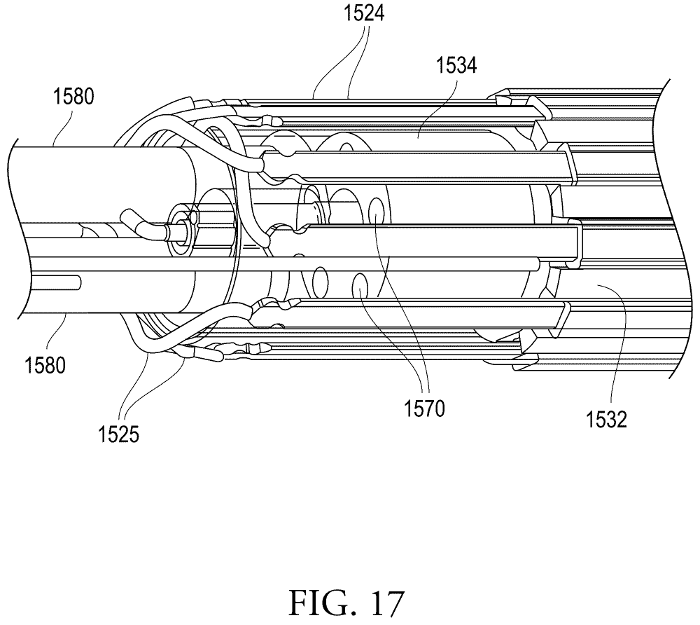

It may be appreciated that desired irrigation fluid flow would be sufficient to reach all or a majority of the potentially stagnant blood-filled areas around the splines 1524. In some embodiments, this is achieved with the use of a plurality of irrigation ports 1570 that are configured to create turbulent flow within the hollow cage of the energy delivery body 1522. Although a single irrigation lumen may provide a large enough output of fluid flow to reach the proximal end of the energy delivery body 1522, the flow may not be strong enough to reach the distal end of the energy delivery body 1522. However, having the single irrigation lumen pass fluid through multiple irrigation ports 1570 creates turbulence in the flow at the proximal end of the energy delivery body 1522 and the turbulent flow continues a wide fan of fluid flow through to the distal end of the energy delivery body 1522. The wide fan also accounts for coverage of the energy delivery body 1522 when the energy delivery body 1522 has been bent or moved laterally during positioning or manipulation. It may be appreciated that such turbulent flow may also be achieved with the use of multiple irrigation lumens. Typically, the number of irrigation lumens is less than the number of irrigation ports so as to deliver adequate flow while creating turbulence. FIG. 17 provides a close-up illustration of a portion of the treatment catheter 1502 within the distal end 1506 of the shaft 1504. Here, the shaft 1504 is removed to reveal the shaft plug 1532 with splines 1524 disposed therearound. Conduction wires 1525 are shown connected with each spline 1525. The conduction wires 1525 extend along the shaft 1504 in the proximal direction for connection with the generator 108 so as to deliver the energy to the splines 1525. The embodiment of FIG. 17 includes two irrigation lumens 1580 that deliver fluid to the irrigation ports 1570. Here, five irrigation ports 1570 are present. It may be appreciated that a variety of irrigation lumens 1580 may be present, including one, two, three, four, five, six or more. Likewise, a variety of irrigation ports 1570 may be present, including one, two, three, four, five, six, seven, eight, nine, ten, eleven, twelve or more. However, for turbulent flow, the irrigation ports 1570 typically outnumber the irrigation lumens 1580.

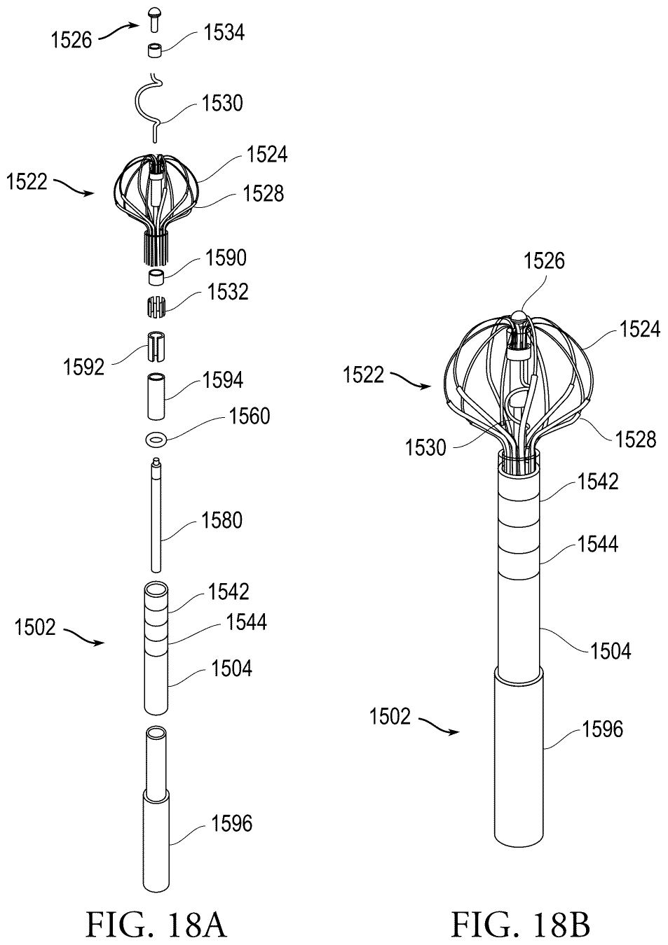

FIGS. 18A-18B provide additional illustrations of the embodiment of FIG. 13. FIG. 18A provides an expanded illustration of elements comprising this embodiment of the treatment catheter 1502. As shown, this embodiment includes a distal tip electrode 1526, a tip inner 1534, a tip electrode wire 1530, an energy delivery body 1522 comprising a plurality of splines 1524 at least partially covered by insulative material 1528, a retention band 1590, a shaft plug 1532, a solder plate 1592, a glue potting 1594, a pull-ring 1560, an irrigation lumen 1580, a shaft 1504 with electrodes 1542, 1544, and a shaft tip section 1596. FIG. 18B illustrates the treatment catheter 1502 of FIG. 39A in its unexpanded state.

As mentioned previously, the treatment catheter 1502 is described as a focal therapy device designed to create lesions that are larger than the footprint of a solid tipped treatment catheter, such as illustrated in FIGS. 2A-2B, yet smaller than the footprint of a one-shot device. In some embodiments, the shaft 1504 of the treatment catheter 1502 is 8 French (2.67 mm) and is delivered with the use of an 8.5 French (2.83 mm) sheath. In such embodiments, the energy delivery body 1522 is configured to fit within the 8.5 French sheath in its collapsed configuration, thus having an outer diameter of less than 2.83 mm. When the energy delivery body 1522 is released to its expanded state, the outer diameter typically expands to 8-15 mm which is 3-6 times the diameter of the shaft 1504. It may be appreciated that the footprints of such devices may vary in size within this range including 8 mm, 9 mm, 10 mm, 11 mm, 12 mm, 13 mm, 14 mm, 15 mm, 8-10 mm, 9-10 mm, 9-15 mm, 10-15 mm, 12-15 mm, etc.

### Sensors and Irrigation

The tissue modification systems 100 described herein deliver a series of PEF batches or bundles described herein over a period of time, such as several seconds. This accumulation of energy deposition results in a small amount of joule heating which is inherent to all PEF therapies as it is a byproduct of energy deposition. However, acute, subacute, medium-, and long-term histological data all indicate that there are no substantial indication of thermal damage to the tissue using the systems, devices and methods described herein. Therefore, it is evident that thermal damage (extracellular protein denaturation) is not generated in the cardiac tissue, reducing the chances of adverse events and anatomical deficits such as pulmonary vein stenosis resulting from the treatment. This also eliminates the generation of surface char or thermal injury which impedes energy delivery to underlying tissue, reducing the ability to generate transmural lesions.

However, it may be appreciated that, in some embodiments, the system 100 includes temperature sensing and/or control measures for various purposes. In some embodiments, temperature is sensed and controlled to ensure that the temperature remains in the range of 30-65° C., 30-60° C., 30-55° C., 30-50° C., 30-45° C., 30-35° C. Thus, lesions are not created by thermal injury as the temperature of the tissue remains below a threshold for thermal ablation. In some embodiments, one or more temperature sensors are used to measure electrode and/or tissue temperature during treatment to ensure that energy deposited in the tissue does not result in any clinically significant tissue heating. For example, in some embodiments, a temperature sensor monitors the temperature of the tissue and/or electrode, and if a pre-defined threshold temperature is exceeded (e.g., 65° C.), the generator alters the algorithm to automatically cease energy delivery or modifies the algorithm to reduce temperature to below the pre-set threshold. For example, in some embodiments, if the temperature exceeds 65° C., the generator reduces the pulse width or increases the time between pulses and/or packets (e.g., delivering energy every other heart beat, every third heart beat, etc.) in an effort to reduce the temperature. This can occur in a pre-defined step-wise approach, as a percentage of the parameter, or by other methods. It may be appreciated that temperature sensors may be positioned on electrodes, adjacent to electrodes, or in any suitable location along the distal portion of the catheter. Alternatively or in addition, sensors may be positioned on one or more separate instruments.

In other embodiments, temperature is sensed to assess lesion formation. This may be particularly useful when generating lesions in anatomy having target tissue areas of differing thicknesses. A rapid rise in temperature indicates that the lesion has penetrated the depth of the tissue and is nearing completion. Sensing such changes in temperature may be particularly useful when generating lesions in thicker tissues or tissues of unknown depth.

In some embodiments, the treatment catheter includes irrigation to assist in controlling the temperature of the delivery electrode or surrounding tissue. In some instances, irrigation cools the delivery electrode, allowing more PEF delivery per time without increasing any potential heat-mediated damage. In some instances, irrigation also reduces or prevents coagulation near the tip of the catheter. It may be appreciated that irrigation may be activated, increased, reduced or halted based on information from one or more sensors, particularly one or more temperature sensors.

Such cooling is achieved by delivering fluid, such as isotonic saline solution, through a lumen in the catheter that exits through one or more irrigation ports along the distal end of the catheter. The fluid may be chilled fluid, room temperature fluid or warmed fluid. The fluid flow can be driven by a variety of mechanisms including a gravity driven drip, a peristaltic pump, a centrifugal pump, etc. In some embodiments, the irrigation has a flow rate of 0.1-10 ml/min, including 1 ml/min, 2 ml/min, 3 ml/min, 4 ml/min, 5 ml/min or more. In some embodiments, the flow rate is sensed by electrical or mechanical flow sensing mechanisms. In some embodiments, the temperature of the fluid is measured, and in other embodiments the temperature of the fluid is modified, such as warmed or cooled, as it is pumped into the treatment catheter, such as based on the measured temperature. In some embodiments, the fluid flow rate is determined based on the measured temperature of the tissue to be treated.

In some embodiments, the pump is in electrical communication with the generator 108 wherein the fluid flow rate is modified by the generator 108 based on the status of energy delivery to the treatment catheter 102. For example, in some embodiments, fluid flow rate is increased during energy delivery. Likewise, in some embodiments, fluid flow rate is increased a predetermined amount to time prior to energy delivery and/or at a predetermined time(s) during energy delivery. Alternatively or in addition, fluid flow may be controlled on demand by the user. It may be appreciated that the pump may communicate with the generator 108 to operate at different speeds based on various aspects of the energy delivery algorithm 152. In some embodiments, sensing of flowrate and communication with the generator 108 is used to prevent energy delivery if irrigation is not running. In other embodiments, selection of an energy delivery algorithm 152 in turn selects a fluid flow rate appropriate for the energy delivery algorithm 152. In some embodiments, at least one irrigation port is located along an electrode and/or optionally at least one irrigation port is located along the shaft.

It may be appreciated that any of the catheter designs described herein may include one or more sensors (e.g., microsensors), such as impedance sensors, contact sensors, contact force sensors, electroanatomic mapping sensors, etc. Such sensors may be positioned on electrodes, adjacent to electrodes, or in any suitable location along the distal portion of the catheter. For example, microsensors may be located along one or more loops of a delivery electrode or along a support structure near the delivery electrode. Alternatively or in addition, sensors may be positioned on one or more separate instruments.

It may also be appreciated that although a variety of the delivery electrodes have been described as conductive wires capable of delivering energy therefrom, such designs may utilize individual electrodes (e.g., microelectrodes) spaced along a non-conductive wire. Optionally, such electrodes may be spaced along a conductive wire when the conductive wire is insulated from the electrodes where the electrodes are attached.

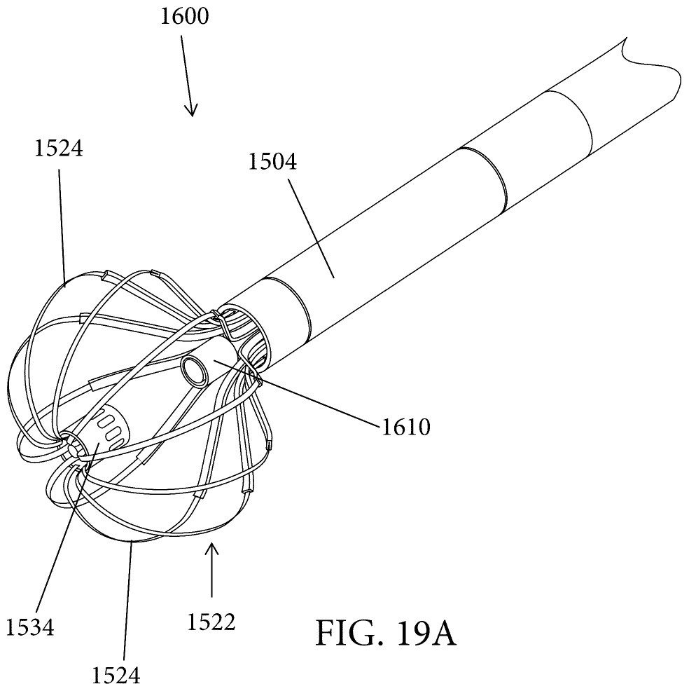

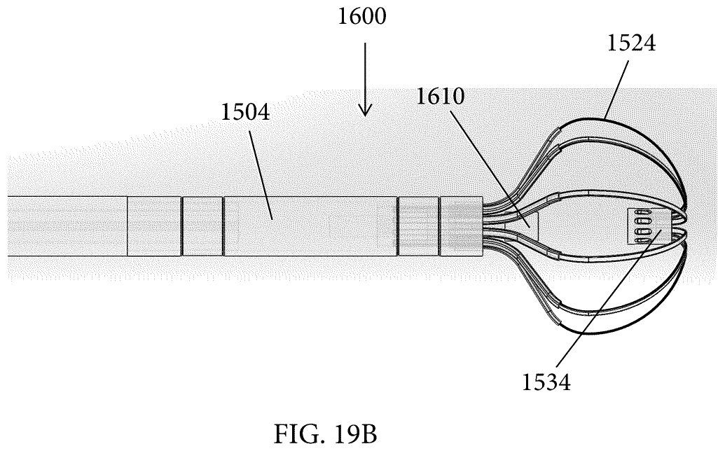

FIGS. 19A and 19B illustrate a treatment catheter 1600 in accordance with another embodiment. The treatment catheter 1600 shares similarities with the treatment catheter 1500 and, therefore, like elements are numbered alike. In particular, the treatment catheter 1600 includes the energy delivery body 1522 defined at least in part by the plurality of conductive splines 1524. In this embodiment, the distal tip electrode 1526 is not present and likewise, there is no tip electrode wire 1530 that is disposed and extends internally within the sphere shaped arrangement of splines 1524. The sphere of the treatment catheter 1600 is thus defined by the expanded plurality of splines 1524.

Instead, at the base of the sphere, there is a NAV (navigation) sensor 1610 that is in the form of a protrusion that extends from the distal end of the shaft 1540 into the lower (proximal) portion of the sphere. As shown, the NAV sensor 1610 is coaxial to the tip inner 1534 but separated and spaced apart therefrom. Since the NAV sensor 1610 takes the place of the sensing electrode 1540 that is coupled to the tip inner 1534 at the upper (distal) portion of the sphere, there is no tip electrode wire 1530 that extends through the interior of the sphere.

As is known, the NAV sensor 1610 can be any number of commercially available NAV sensors that each is designed to provide improved navigation of the treatment catheter 1600. For example, the NAV sensor 1610 can be in the form of a magnetic navigation sensor that provides precise catheter location.

The embodiment shown in FIGS. 19A and 19B utilizes a NAV sensor 1610 in contrast to the distal tip electrode 1526 shown in FIG. 13. However, it will be appreciated that like the distal tip electrode 1526, the NAV sensor 1610 acts as a central electrode that is internal to the basket. As mentioned, the NAV sensor 1610 is at a proximal location compared to the distal tip electrode 1526; however, in both embodiments (i.e., the distal tip electrode 1526 and NAV sensor 1610 embodiments), contact is determined by applying a small current from the central electrode (NAV sensor 1610) (center of basket) to one or more of the electrodes (spline electrodes) on the sphere. Alternatively, in both configurations, current can be applied from one electrode (one spline electrode) on the sphere (spline) to another electrode (another spline electrode). Both approaches provide the user with contact information but the sensitivity is different from spline electrode to spline electrode than it is from the reference or central electrode to spline electrode.

### Balloon Treatment Catheter

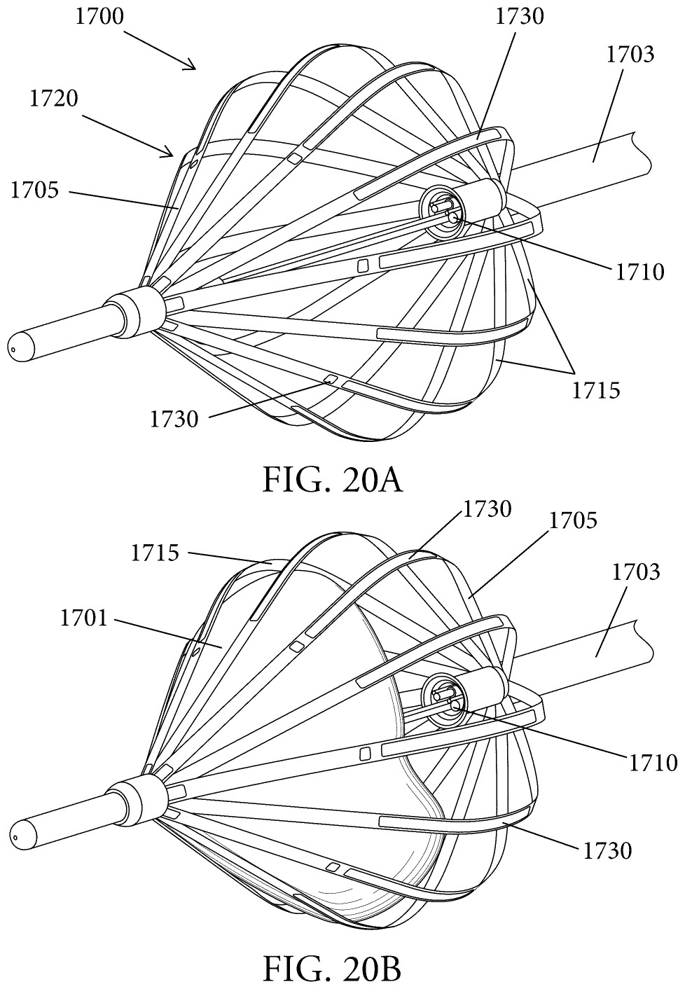

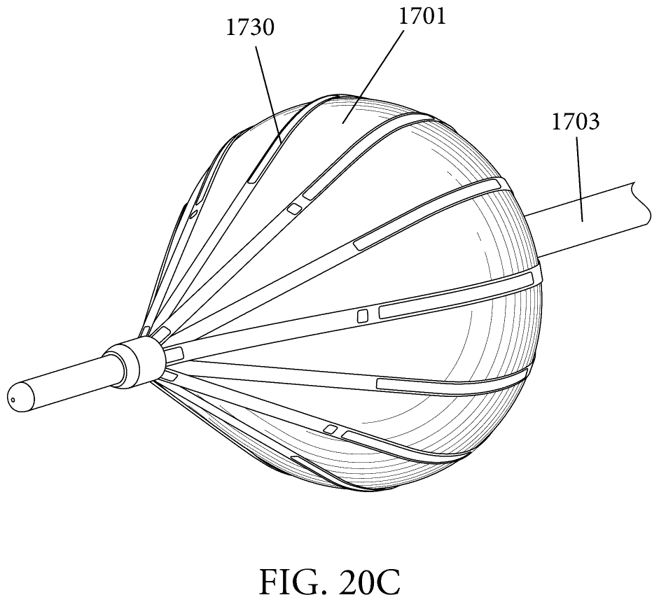

Now referring to FIGS. 20A-20C, in which another treatment catheter 1700 in accordance with another embodiment is shown. Unlike the other catheters described and illustrated herein, the treatment catheter 1700 comprises a balloon catheter.

The treatment catheter 1700 is similar to that disclosed in commonly owned US patent application publication No. US2022/0323143, which is hereby expressly incorporated by reference in its entirety. More specifically, the treatment catheter 1700 can be used for energy delivery and an exemplary method for ablating target tissue includes the steps of: (a) delivering treatment catheter 1700 to the target tissue, wherein the treatment catheter 1700 includes a compliant balloon 1701, a visualization device 1710; and an electrode array 1720 that is visible to the visualization device 1710, each electrode being configured to deliver ablation energy, wherein the electrode array 1720 can be independently movable relative to the compliant balloon 1701; (b) isolating the target tissue such that at least one electrode of the electrode array is in contact with the target tissue; and (c) delivering the ablation energy to those electrodes of the electrode array 1720 that are confirmed, using the visualization device 1710, to be in contact with target tissue.

As with the other embodiments disclosed herein, the treatment catheter 1700 is part of or integrated with a computer implemented system, which can be configured with one or more processors (e.g., processor 154), memory, controller, and a display, for example to process data received from connected devices into visual information. The visualization device 1710 is preferably in the form of an endoscope that is preferably provided in treatment catheter 1700 and can be configured with a lens, an image sensor, and memory for capturing and recording both live images and still images. The location of the endoscope is discussed further herein. The processor(s) can be configured, for example, by executing instructions stored on non-transitory processor readable media, to process information of various types and from various sources, including live visual data and data provided via one or more of ablation sensors, instrument controllers, and display(s).

In one or more embodiments, the one or more processors can be configured by executing instructions, for example, provided in a series of software and/or hardware modules, to interpret, manipulate and record visual information received from the treatment site. Moreover, the processors can be configured to manipulate and provide illustrative and graphical overlays, and generate composite or hybrid visual data to the display device. The ablation system can further include haptic technology that provides vibratory or other feedback in response to information processed by one or more processors. For example, as described herein, a display provides a graphical representation of an impedance indicator, as a sum of electrode inputs. In addition to a visual, graphical representation, the ablation system can include a catheter configured to provide haptic feedback that corresponds to the visual impedance representation. Thus, the ablation system can include multiple information provisioning, including visual and physical feedback and substantially in real-time.

The ablation system that includes the treatment catheter 1700 can further be configured with a user interface that is operatively coupled to the processor(s) and/or controller. For example, the controller can be configured to control the output of the energy source, the illumination and excitation sources of an energy transmitter, as well as to process information to determine distance and movement of an energy transmitter relative to tissue at an ablation treatment site (as discussed further below). As will also be appreciated from the below discussion, an endoscope is preferably supported by the treatment catheter 1700 and captured images can be processed by the processor(s), including to determine whether sufficient ablative energy deliveries have been directed to a specific area of a treatment site. As noted herein, data obtained from the endoscope can include video and still images of the treatment site captured substantially in real-time and as seen from the ablation instrument. Video and still images can be stored in memory for later use.

The treatment catheter 1700 includes a main catheter shaft 1703 that has a distal end. It will also be appreciated that the treatment catheter 1700 can include more than one shaft and often includes an inner catheter shaft and an outer catheter shaft or can otherwise include multiple concentric tubular structures. An inflatable balloon 1701 is included and is coupled to the main catheter shaft 1703 with a distal end of the inflatable balloon 1701 being proximate the distal end of the main catheter shaft 1703 and the proximal end of the inflatable balloon 1701 being spaced from the distal end. The inflatable balloon 1701 thus surrounds the main catheter shaft 1703. FIG. 20B shows the balloon 1701 partially broken away to show the inside components and FIG. 20C shows the balloon 1701 inflated.

The treatment catheter 1700 can include an inner shaft along with an endoscope (the visualization device 1710). The endoscope extends along the exterior of the inner shaft and is typically located at one end of the balloon is forward looking in that it looks forward toward the other end of the balloon.

The endoscope is located within the compliant balloon 1701. The endoscope allows the operator of the treatment catheter 1700 to visualize the treatment area and the progress of lesion formation. Such a system is described in Melsky et al. (U.S. Pat. No. 9,421,066 (the '066 patent) and Melsky et al. (U.S. Pat. No. 9,033,961 (the '961 patent), each of which is incorporated by reference in its entirety. Accordingly, the endoscope is at a location that is proximal to the location at which the energy is delivered to the tissue to allow the user to view the delivery of the energy and the resultant tissue lesion(s). The endoscope can be one of the ones described herein and also one that is described in any one of the documents incorporated by reference herein.

The endoscope is forward-facing and is disposed adjacent to one of the catheter shafts, such as a central tubing typically formed of a transparent polymer material. As used herein, the term forward-facing refers to the view of the endoscope in a distal direction relative to the catheter body. Similarly, the term side-facing refers to the view of the endoscope in a direction that is radially outward from a side of the catheter body. The endoscope can be a fiber optic endoscope that is inserted through a lumen of the catheter and located within a proximal region of the inflatable balloon. In another embodiment, the treatment catheter 1700 includes first and second imaging devices for providing direct visualization of the region to be treated, with the first imaging device being fixed relative to the catheter body. The first and second imaging devices can be in the form of first and second imaging chip endoscopes. Details of the first and second imaging chip endoscopes are described in commonly owned U.S. patent application Ser. No. 17/524,472, which is expressly incorporated herein by reference in its entirety.

In one or more embodiments, the treatment catheter 1700 includes an expandable basket 1705 that surrounds the inflatable balloon 1701 and is configured to expand upon expansion (inflation) of the inflatable balloon and similarly, is configured to contract upon deflation and contraction of the inflatable balloon. The expandable basket 1705 can have a first collar (first ring) at a first (proximal) end of the expandable basket 1705 and a second collar (second ring) at a second (distal) end of the expandable basket 1705. The first and second collars have annular shapes and can thus have a continuous ring shape. The size of the two collars can be different from one another with the first collar in the illustrated embodiment being larger than the second collar. The two collars are sized and configured to fixedly couple the expandable basket 1705 to the main catheter shaft (or one or more other catheter shafts) with the inflatable balloon 1701 being located between the two collars. The first collar is thus preferably located proximal to the inflatable balloon 1701, while the second collar is located distal to the inflatable balloon.

The expandable basket 1705 includes a plurality of splines 1715 that are attached at one end to the first collar and at the other end to the second collar. The plurality of splines extend longitudinally along a length of the inflatable balloon. The plurality of splines 1715 are circumferentially offset from one another with open spaces formed between adjacent splines 1715. The splines 1715 are constructed to expand and contract under action of the underlying inflatable balloon 1701. In particular, when the inflatable balloon 1701 expands under inflation, the splines 1715 expand outwardly and conversely, when the inflatable balloon 1701 contracts under deflation, the splines 1715 contract inwardly. The splines 1715 thus conform to the shape of the inflatable balloon 1701.

Each spline 1715 carries one or more electrodes 1730. For example, each spline 1715 can include a plurality of electrodes 1730 that can be configured as an electrode array. In the illustrated embodiment, there are three electrodes 1730 located along the length of the spline 1715. The electrodes 1730 are spaced longitudinally along the spline (in series). The electrodes 1730 are thus spaced apart from one another a predefined set distance. The locations of the splines 1715 are selected so as to centrally position the electrodes 1730 relative to the inflatable balloon 1701 since when the inflatable balloon 1701 is inflated, the electrodes 1730 are, as discussed herein, for placement against the target tissue to be ablated using PFA (pulsed field ablation) technique.

The electrodes 1730 that define the electrode array can be the same electrode type or they can be different. For example, the shapes and sizes of the electrodes 1730 can be the same as shown. The material of the expandable basket is not elastic in that the splines do not stretch elastically in a longitudinal direction but can expand and contract with the underlying inflatable balloon. Thus, the longitudinal spacing between the electrodes does not change when the expandable basket moves between the expanded position and the retracted position. Instead, it is a fixed distance which is important and this information is used during the visualization and ablation process in order to form the desired lesion as discussed herein.

The embodiment illustrated in FIGS. 20A-20C thus uses a visualization device for guidance which can be used in combination with the local impedance indicator disclosed herein to provide information to the user concerning the progress and status of the lesion.

In the treatment catheter 1700, the local impedance can be measured either electrode to electrode around the ballon diameter or the present system can measure impedance from the ballon electrode to a reference electrode positioned on the shaft of the catheter or on the distal end of the balloon catheter, similar to the previous embodiments described herein. The use of a reference electrode with the treatment catheter 1700 thus acts similar to the center electrode (e.g., distal tip electrode 1526) in that the local impedance can be measured between one spline electrode and this reference electrode.

### Display

As mentioned, generally, a tissue modification system can include a specialized treatment catheter, a high voltage waveform generator and at least one processor configured to apply a distinct energy delivery algorithm. Additional accessories and equipment can be utilized, including an electroanatomic mapping (EAM) system. EAM systems allow operators to generate anatomic geometry of the heart and record intracardiac electrical activation in relation to specific anatomic locations within the heart by capturing measured electrical signal metrics (intensity, delay of signal arrival from a distant stimulus, etc.). Thus, the EAM technology allows one to accurately determine the location of arrhythmia origin, define cardiac chamber geometry in three dimensions, delineate areas of anatomic interest, and allow catheter manipulation and positioning without fluoroscopic guidance. In addition, devices, systems and methods described herein provide information related to the effectiveness of the treatment during the procedure so as to create an electrical blockade within the heart that will remain durable and effective long-term. In one or more embodiments, information via EAM technology is generated with the use of a mapping catheter and in other instances this is achieved with technology built into the specialized treatment catheter. One or more embodiments include EAM technology combined in the specialized treatment catheter although other configurations, including a mapping catheter and a treatment catheter, are supported and envisioned herein.

EAM systems are available in various forms, including CARTO®, ENSITE™ systems, KODEX-EPD systems, ACQMAP systems, and RHYTHMIA HDX™. The CARTO mapping system, for example, utilizes a low-level magnetic field delivered from three separate coils in a locator pad beneath the patient. The magnetic field strength from each coil is detected by a location sensor embedded proximal to the tip of a specialized treatment catheter. The strength of each coil's magnetic field measured by the location sensor is inversely proportional to the distance between the sensor and coil. Hence, by integrating each coil's field strength and converting this measurement into a distance, the location sensor (and, therefore, catheter tip location) can be triangulated in space. The specialized treatment catheter typically includes proximal and distal electrode pairs, and a tip electrode capable of treatment energy delivery. The catheter can be moved along a surface of the heart to record local endocardial activation times for arrhythmia mapping, while simultaneously recording location points to generate three-dimensional (“3D”) chamber geometry. An accurate representation of chamber geometry can be provided, as well as isochronal activation maps and playable propagation maps. Further, locations of important anatomic landmarks can be recorded (e.g., the bundle of His), areas of electrical scar (e.g., to create voltage/scar maps), and vessels (e.g., coronary sinus, pulmonary veins). Moreover, EAM systems, such as the CARTO mapping system, can record an ablation lesion location, thereby facilitating creation of ablation lines.

Prior to arrhythmia mapping, a stable location reference can be established, for example, by placing a location magnet (e.g., a triangular apparatus containing three magnetic coils) beneath the patient and table. The location of the location magnet can be aligned anywhere inside a defined circumference at the start of the procedure. Further, a reference patch can be affixed to the patient's back roughly overlying the cardiac chamber of interest. Should the location reference magnet or patch become displaced during the procedure, their original location can be recorded to allow proper repositioning. This allows for accurate tracking of mapping catheter position, consistency of anatomic landmark and ablation lesion locations, and precise reconstruction of chamber geometry.

Once the location reference has been stably situated, an appropriate timing reference and window is selected. The timing reference can be any selected recording, such as intracardiac electrogram (EGM) or surface ECG lead, representative of activation of the chamber of arrhythmia origin. Intracardiac EGMs are often selected as the timing reference as these are generally more consistent in appearance and precise in timing than surface ECG recordings, and consequently, are more reliable. Any component of the reference electrogram may be chosen for a timing reference, including maximum (peak positive) deflection, minimum (peak negative) deflection, maximum upslope (dV/dT) or maximum downslope. In addition to using an electric signal metric based on a timing within the incoming signal, the time delay for the electrical signal itself to reach the location of interest from a natural or induced electrical propagation event may also be measured.

In addition to facilitating activation mapping, the EAM system provides location mapping features capable of recording sites of anatomic relevance, areas of low endocardial voltage representing scar, and areas of ablation. Ablation causes a variety of cellular reactions, as will be described in more detail herein, ranging from stunning to dying and death. Cardiomyocytes in each of these stages do not conduct electricity almost immediately after ablation. Stunned cardiomyocytes recover and will regain the ability to conduct electricity, a process that occurs over minutes to hours or longer. Dead and dying cardiomyocytes will cease to conduct electricity over the long term. Over the 1-4 weeks following ablation, the dead cells are removed by the body and replaced by scar tissue, and the accumulated dead zones are used to create durable conduction blockades. Targeted specific anatomic locations include the superior vena cava, inferior vena cava, right pulmonary vein, left pulmonary vein, right atrium, right atrial appendage, left atrium, left atrial appendage, right ventricle, left ventricle, right ventricular outflow tract, left ventricular outflow tract, ventricular septum, left ventricular summit, regions of myocardial scar, myocardial infarction border zones, myocardial infarction channels, ventricular endocardium, ventricular epicardium, papillary muscles and the purkinje system, to name a few. Treatments are delivered at isolated sites or in a connected series of treatments. Types of treatment include the creation of left atrial roof line, left atrial posterior/inferior line, posterior wall isolation, lateral mitral isthmus line, septal mitral isthmus line, left atrial appendage, right sided cavotricuspid isthmus (CTI), pulmonary vein isolation, superior vena cava isolation, vein of Marshall, lesion creation to target Complex Fractionated Atrial Electrograms (CFAE), lesion creation to target Focal Impulse and Rotor Modulation (FIRM), and targeted ganglia ablation. Such tissue modification creates a conduction block within the tissue to prevent the transmission of aberrant electrical signals.

In one or more embodiments, tissue modification is achieved by treating tissue in a point-by-point fashion with the use of a treatment catheter 102. As mentioned, the overall system described herein can include a display of an EAM system while treating cardiac tissue of the heart in a point-by-point fashion with the use of the treatment catheter 102 (with assistance of mapping) to create a treatment zone of overlapping lesions. The lesions can be displayed as spheres in 3D space.

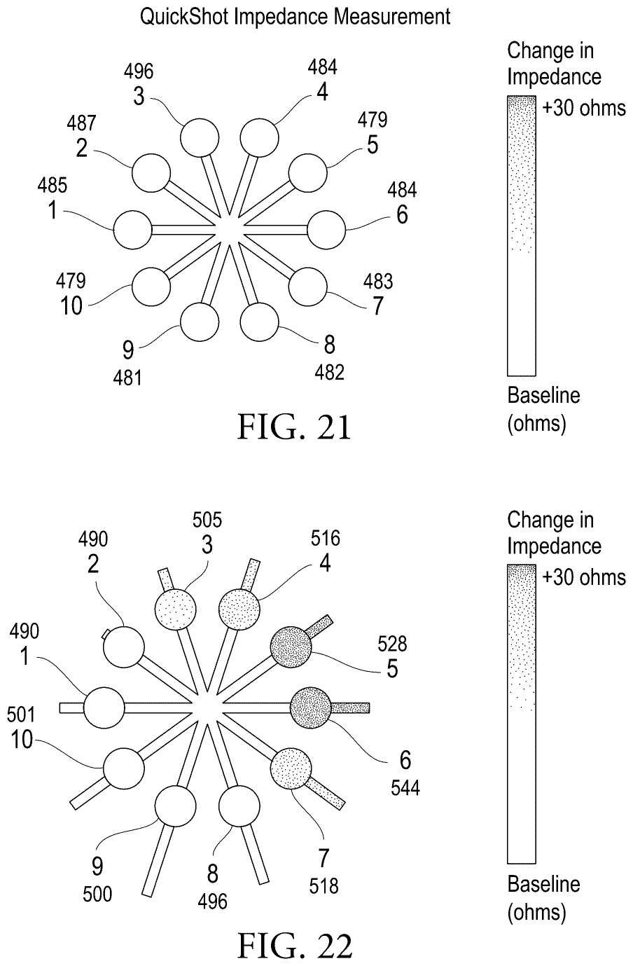

The display also can provide a local impedance indicator in the lower right corner of the display. The local impedance indicator, which is described in more detail below and illustrated in FIGS. 21-22 with reference character 2000, displays relative changes in impedance to the user in order to quickly communicate information that helps the user better understand where in the heart chamber the catheter is located. In this embodiment, the local impedance indicator 2000 has a shape that correlates to the end effector of the treatment catheter, particularly the view of the end effector illustrated in FIG. 15. FIG. 15 illustrates a bottom view of the treatment catheter 1502 of FIGS. 13-14, facing the convex distal face of the energy delivery body 1522. Here, the energy delivery body 1522 includes ten splines 1524. Thus, in this embodiment, the local impedance indicator 2000 has a wheel shape having ten spokes, wherein each of the spokes of the wheel represent a spline and end in a circle that represents the electrode. There are ten spokes to match the ten splines. It may be appreciated that the number of spokes of the local impedance indicator 2000 may vary so as to match the number of splines. Alternatively, particular spokes may be visually reduced, such as greyed out, if they do not correlate to a spline on the catheter.

FIG. 21 provides an enlarged view of the local impedance indicator 2000. In operation, spokes of the local impedance indicator 2000 can change properties, such as color, color intensity, shade, hue, saturation, brightness, or other color characteristics as the local impedance changes during the procedure. For sake of illustration, in the figures the stippling gradient reflects a change in color and/or intensity. A greater density of stippling indicates a greater impedance value (in ohms). The local impedance is measured between each spline electrode 1524 and the center (distal) electrode 1526. In FIGS. 21 and 22, the individual electrodes are numbered consecutively for reference purposes and a measured impedance value is listed (in ohms) for each individual electrode. The impedance values in FIG. 21 are thus baseline values.

When the user first inserts the treatment catheter into the heart chamber, the catheter is navigated to the middle of a blood pool to “zero” the program. This causes all of the spokes/electrodes of the local impedance indicator 2000 to show a baseline color, typically a neutral color such as a grey color (depicted in FIG. 21 with no color (white) for illustration purposes). This state of the local impedance indicator 2000 is illustrated in FIG. 21. Each electrode also has a baseline value associated with it at this time.

Referring to FIG. 22, the local impedance indictor 2000 changes color and/or shape as the sensed local impedance values change to provide quick graphical feedback to the user. The local impedance indicator 2000 is associated with a threshold value in which sensed local impedance changes from baseline to a value. Once again, the numeric values listed within or next to each spline electrode is the measured impedance value in real time. When the sensed local impedance crosses a threshold value, the local impedance indictor 2000 changes color, such as from grey to green, along the corresponding spoke. As the sensed local impedance increases, the spoke extends or “grows” away from the center so as to provide a visual representation of the increase in impedance intensity. The local impedance indicator 2000 is also associated with one or more range values, wherein when the sensed local impedance crosses the one or more range values, the intensity of the color changes, such as through various shades from light green color to dark green color along with corresponding extension of the spoke reaching maximum extension at the dark green color. As the end effector of the treatment catheter moves around the heart chamber, the local impedance indicator 2000 shows the change in sensed impedance corresponding to the splines of the end effector by changing the intensity of the color of the corresponding spokes (e.g., change in color to the corresponding green of increased intensity) and length of the spokes. Thus, good, solid tissue contact can quickly and easily be found on the display by finding the darker and/or longer spokes.

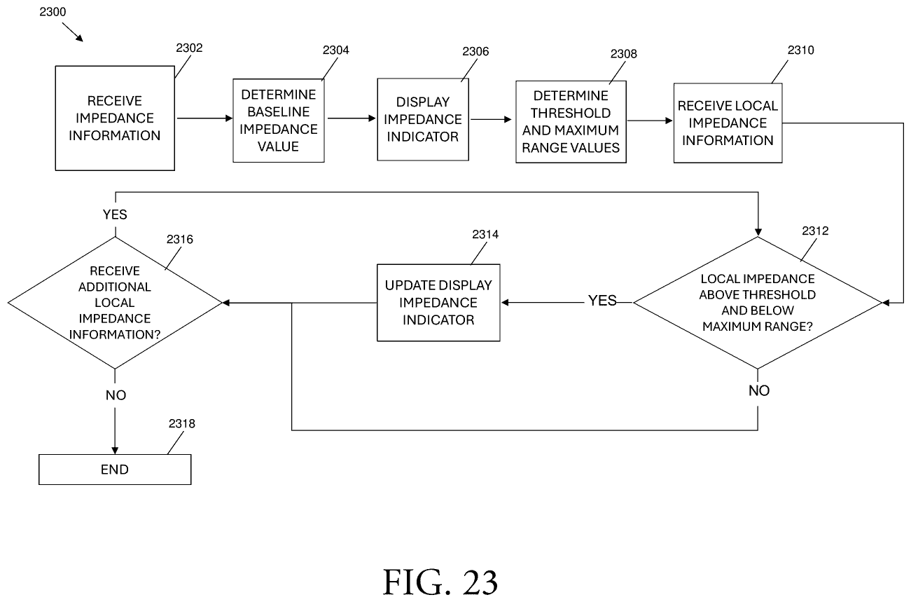

FIG. 23 is a flow chart illustrating example steps 2300 that are associated with preprocessing in accordance with an example implementation of the present disclosure. It should be appreciated that several of the logical operations described herein are implemented (1) as a sequence of computer implemented acts or program modules running on a communication device and/or (2) as interconnected machine logic circuits or circuit modules within a communication device. The implementation is a matter of choice dependent on the requirements of the device (e.g., size, energy, consumption, performance, etc.). Accordingly, the logical operations described herein are referred to variously as operations, structural devices, acts, or modules. Several of these operations, structural devices, acts and modules can be implemented in software, in firmware, in special purpose digital logic, and any combination thereof. It should also be appreciated that more or fewer operations can be performed than shown in the figures and described herein. These operations can also be performed in a different order than those described herein. More specifically, in another embodiment, the change in impedance value from the baseline values can use a graphic gradient scale that goes from red (showing no change and thus, no contact) to different shades of green showing different levels of satisfactory contact, with for example, a dark shade of green indicating strong contact.

In the example steps shown in FIG. 23, the process begins at step 2302 and impedance information is received, for example, from an electrode (e.g., electrode 1 of 10 electrodes arranged in a spline). Using the impedance information received in step 2302, a baseline impedance value can be determined (step 2304). For example, electrode 1 is “zeroed” and the baseline value is 50 ohms. At step 2306, the display indicator 2000 is displayed, in which the display indicator is formatted in accordance with the baseline value (e.g., in a neutral grey color and the spokes are all equally long). Thereafter, at step 2308, a threshold impedance value and a maximum range value is set, for example, to 10 ohms and a maximum range value is set, for example, to 30 ohms. As the electrode comes into contact with tissue, the sensed local impedance changes (i.e., rises). Information associated with local impedance, such as a value representing local impedance as electrode 1 comes in contact with tissue, is received (step 2310). At step 2312, a determination is made whether the local impedance exceeds the threshold value (e.g., beyond neutral) and whether the local impedance exceeds the maximum range value. If the determination is affirmative, then the process branches to step 2314 and the impedance indicator is updated and displayed. For example, when electrode 1 rises in impedance to 60 ohms (+10 ohms from baseline), the corresponding electrode of the local impedance indicator 2000 is altered to display a light green color and the spoke will increase in length (e.g., a bar will grow beyond the circle representing the electrode). The color will continue to darken to darker green and the bar will continue to grow as the sensed local impedance grows. When the local impedance of electrode 1 is 80 ohms (+30 ohms from baseline), the corresponding electrode of the local impedance indicator 2000 will be the darkest color green and the bar will be fully extended.

Continuing with reference to FIG. 23, if the determination at step 2312 is negative, then the process continues to step 2316 and a determination is made whether additional local impedance information is received, thereby requiring further updates to the impedance indicator 2000. Similarly, the process continues from step 2314 to step 2316. If the determination at step 2316 is affirmative, then the process loops back to step 2312 and a further determination is made whether the local impedance value exceeds the threshold value. Alternatively, if the determination at step 2316 is negative, then the process branches to step 2318 and the process ends. For example, therapeutic energy is delivered to the site.

The disclosed system can include additional features such as the following features that can be implemented. The present system can be configured to receive and process measurements from a combination of electrodes to confirm contact of a region of the energy delivery body with tissue. More specifically, the system can use a weighted rating that looks at the one respective electrode plus the adjacent electrodes (spline electrodes) to determine if we can trust the electrode reading (received from one respective spline electrode). For example, if the electrode (spline electrode) in question shows contact but measurements from the adjacent two electrodes (spline electrodes) do not, the present system would determine that the one respective electrode is also not in contact. In other words, the present system would determine that the reading (local impedance measurement) from the one respective electrode is a false positive. Alternatively, if the system calculates that three adjacent electrodes (spline electrodes) all showing contact and the electrodes on the opposite sides of the tip (i.e., opposite sides of the three adjacent electrodes) show contact, then the system calculates that full contact is present.

In one embodiment, the weighted rating can be based on a ratio or threshold comparison. For example, if the local impedance value of the adjacent electrodes is 50% or greater of the impedance value of the one respective electrode, then contact with tissue is assumed. For example, if the impedance value of the one respective electrode is 100 ohms, then if the adjacent electrodes have impedance values of 50 ohms or greater, it is deemed that full contact is present. Conversely, if the adjacent electrodes have impedance values of less than 50 ohms, then the one respective electrode is deemed not to be in full contact.

The present system can also be configured to process electrode measurements to determine proportional generator output (which electrodes and variable energy for a given electrode or group of electrodes (i.e., spline electrodes)). In one embodiment, the system is configured to deliver 33A to the full tip (the energy delivery body comprising the spline electrodes) regardless of number of electrodes in contact with tissue (as determined by local impedance values, visualization and/or other techniques). In one embodiment, the system includes ten electrodes (ten splines) and the system can be configured such that the number of electrodes is proportional to the tip contact area. So with fewer electrodes as part of the system, the system can increase ablation energy to ensure that the current density is the same during energy delivery for any contact scenario. If there were too few electrodes, the system would not allow ablation energy delivery.

For example, if it is determined that if less than a threshold number of the electrodes are in contact with tissue, then the dose will be adjusted in order to provide a dose that achieves the same target depth of the lesion (e.g., 6-8 mm) that is desired. For example, if the system is of a type in which 33 amps is delivered when solid contact is established (e.g., when all spline electrodes in contact) and if it is determined that less than 50% of the electrodes are in contact, then the dose (amps) can be increased to ensure the target depth of lesion is achieved. For example, the amps may be increased to 45-50 amps for the dose in view of less than full contact between the electrodes and tissue.

In yet another feature, the system can also use time analysis of electrode measurements to determine contact stability. This feature ensures that there isn't intermittent contact between the electrodes and the target tissue. For example, by looking at first and second electrodes (e.g., splines electrodes #1 and #2) and comparing the sequence of impedance measurements over time from these electrodes, the present system determines if there is stable contact or not. This would be looking impedance measurement stability on a single electrode and comparing that stability rating to those adjacent electrodes.

For example, for an anatomical location, there can be a threshold stability calculation to determine whether the electrode contact is stable. For example, stability is established when the measured impedance value remains within a range over a predetermined period of time. For example, within the left side of the heart, stability may be an impedance value of 10 ohms +−3 ohms, meaning that if the measured impedance over the predetermined period of time (e.g., 500 milliseconds) remains within 7 ohms to 13 ohms, then stability is recorded. For the right side of the heart, different magnitude of values are measured and thus, stability may be a measured impedance value of 60 ohms +−10 ohms meaning that if measured impedance over the predetermined period of time remains within 50 ohms to 70 ohms, then stability is recorded.

In another embodiment, the impedance indicator described and illustrated herein can have a different configuration. In particular, the impedance indicator can be the sum of all electrode inputs (i.e., the sum of all impedance values from all electrodes (all electrode splines) and is displayed as a vector. In contrast, the spoke/spider orientation shown in FIGS. 21-22 in which an electrode spline comprises one leg of the spoked structure, a directional vector can be provided and shows the region of greatest contact of the energy delivery body as described below.

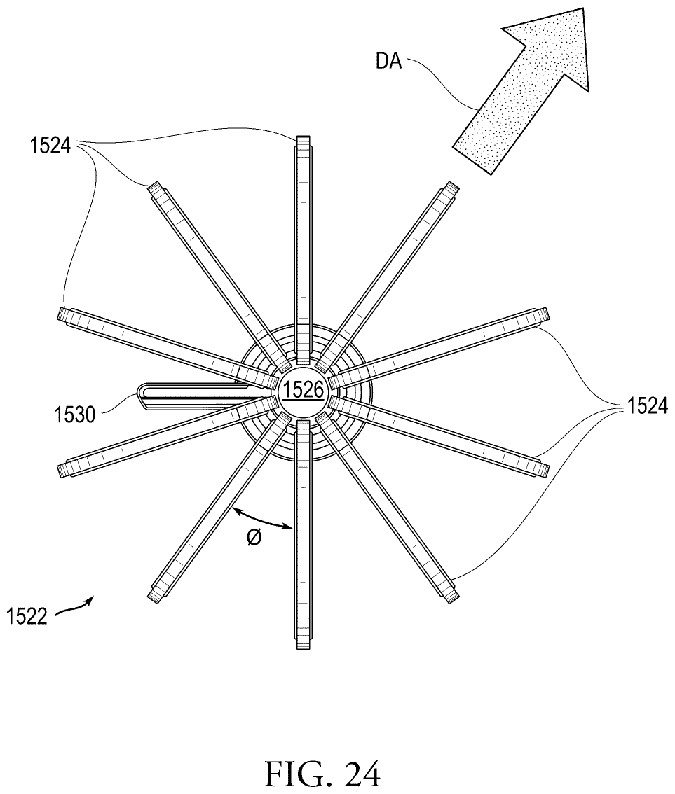

FIG. 24 shows a directional vector (DA) which indicates the direction of the region of the energy delivery body in greatest contact with tissue (based on measured local impedance values described above). It will be appreciated that other graphic information can be provided, such as reference numbers for the electrodes and also if a visualization device is used, images from that device as well. The directional vector is a real time representation and thus, as the treatment catheter moves, the directional vector moves on the display and the graphical information provided thereby, such as the degree of contact changes as well. In addition, similar to FIGS. 21-22, the intensity/magnitude of the impedance values can be indicated graphically, such as by color and/or shading. In this case as in FIGS. 21-22, a greater density of stippling indicates a greater impedance value (in ohms). In the event that all electrode splines are in solid contact with tissue, instead of using a directional area, a graphic in the form of a circle can be displayed around the spokes (spline representations) and similarly, the intensity/magnitude of the impedance can be indicated graphically, such as by color and/or shading of the circle.

In another embodiment, the system is configured such that a vector is sent and displayed on a mapping system as a 3D vector on a moving catheter graphic (such as on FIG. 18B). In other words, on the display in which the tip of the catheter is being observed (e.g., as by use of the visualization device), the vector is displayed and can easily be viewed and provides guidance to the user. In yet another embodiment, the algorithm, executed by the processor is used to determine a summative impedance vector/direction by principal component analysis or similar statistical analysis of time-varying data. This allows for a running display that would show statistical characteristics of the contact like variability, statistical deviation, range, etc. In yet another embodiment, the magnitude of the display is integrated into haptic feedback within the catheter. This feature provides feedback to the customer that gives a slight vibration or pulse in the handle that the user can feel which provides confirmation that sufficient contact is made to start ablating.

For example, the haptic feedback that is provided to indicate that sufficient contact is established and the user should proceed with the energy delivery can be based on different criteria, such as the determination that a threshold number of electrodes are in contact with the tissue; when the measured impedance is above a threshold value; and/or based on time analysis calculation as discussed herein.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system for delivering therapeutic energy during tissue modification treatment, the system comprising:
at least one catheter;
an energy delivery body configured with the at least one catheter;
a plurality of spline electrodes configured with the at least one catheter;
at least one impedance sensor configured with the at least one catheter, each of the at least one impedance sensor respectively associated with at least one of the plurality of spline electrodes;
at least one processor configured by executing instructions stored on processor-readable media to process information associated with the at least one impedance sensor; and
a display that is configured to provide information processed by the at least one processor,
wherein the at least one processor is further configured to:
determine a baseline impedance value, wherein the baseline impedance value is based on impedance sensed by the at least one impedance sensor;
display, on the display, an impedance indicator that is configured with a plurality of spokes, each of the spokes respectively associated with respective ones of the spline electrodes, wherein each of the spokes is configured to represent the baseline impedance value;
define a threshold impedance value;
detect, by the at least one impedance sensor, a local impedance associated with at least one of the spline electrodes navigating via the at least one catheter about an organ;
determining, by the at least one processor, a change in impedance from the baseline impedance to the local impedance;
altering, by the at least one processor, as a function of the change in impedance, at least one respective spoke of the impedance indicator to generate an altered impedance indicator;
displaying, on the display, the altered impedance indicator; and
wherein the tissue modification apparatus delivers the therapeutic energy via the energy delivery body.

2. The system of claim 1, wherein each of the spokes is configured to have a single length to represent the baseline, and
further wherein the at least one processor is configured to alter respective ones of the at least one spoke by extending the length of the respective ones of the at least one respective spoke.

3. The system of claim 1, wherein the at least one processor is further configured to:
define a threshold impedance value,
wherein each of the spokes is configured with a color to represent the baseline, and
further wherein the at least one processor is further configured to alter the at least one spoke by changing the color of the at least one respective spoke when the detected local impedance crosses the threshold value.

4. The system of claim 3, wherein the at least one processor is further configured to:
define a plurality of threshold impedance values, and
alter the at least one spoke by altering the changed color of the at least one respective spoke when the detected local impedance crosses each of the plurality of threshold values.

5. The system of claim 4, wherein the at least one processor is further configured to alter the changed color by altering at least one of the changed color's intensity, shade, hue, saturation, and brightness.

6. The system of claim 1, wherein the at least one catheter further includes a center electrode.

7. The system of claim 6, wherein the detected local impedance is based on impedance sensed by at least one impedance sensor associated with at least one of the plurality of spline electrodes and at least one impedance sensor associated with the center electrode.

8. The system of claim 1, wherein the at least one catheter includes a treatment catheter and a mapping catheter.

9. The system of claim 1, wherein the at least one processor is further configured to:
analyze, as a function of time, electrode measurements to determine contact stability.

10. The system of claim 1, wherein the impedance indicator represents a sum of all electrode inputs in the form of a vector.

11. The system of claim 10, further comprising a mapping component, wherein the at least one processor is further configured to transmit, to the mapping component the vector, and
further wherein the mapping component displays the vector as a three-dimensional vector on a moving catheter graphic.

12. The system of claim 1, wherein the processor is further configured to:
determine a summative impedance vector/direction by principal component analysis or a statistical analysis using time-varying data.

13. The system of claim 1, further comprising haptics configured with the at least one catheter, wherein the processor is further configured to:
provide haptic-based feedback associated with a state of the impedance indicator.

14. A method for delivering therapeutic energy during tissue modification treatment, the method comprising:
determining a baseline impedance value, by a tissue modification apparatus that includes at least one catheter, an energy delivery body, at least one impedance sensor, at least one processor, and a display, wherein the at least one catheter includes a plurality of spline electrodes;
displaying, on the display, an impedance indicator that is configured with a plurality of spokes, each of the spokes respectively associated with respective ones of the spline electrodes, wherein each of the spokes is configured to represent the baseline impedance value;
defining, by the at least one processor, a threshold impedance value;
detecting, by the at least one impedance sensor, a local impedance associated with at least one of the spline electrodes navigating via the at least one catheter about an organ;
determining, by the at least one processor, a change in impedance from the baseline impedance to the local impedance;
altering, by the at least one processor, as a function of the change in impedance, at least one respective spoke of the impedance indicator to generate an altered impedance indicator;
displaying, on the display, the altered impedance indicator; and
delivering, by the tissue modification apparatus, therapeutic energy.

15. The method of claim 14, wherein each of the spokes is configured to have a length to represent the baseline, and
further wherein altering the at least one spoke comprises extending the length of the at least one respective spoke.

16. The method of claim 14 further comprising:
defining, by the at least one processor, a threshold impedance value,
wherein each of the spokes is configured with a color to represent the baseline, and
further wherein altering the at least one spoke comprises changing the color of the at least one respective spoke when the detected local impedance crosses the threshold value.

17. The method of claim 16, further comprising defining, by the at least one processor, a plurality of threshold impedance values,
further wherein altering the at least one spoke comprises altering the changed color of the at least one respective spoke when the detected local impedance crosses each of the plurality of threshold values.

18. The method of claim 17, wherein altering the color comprises:
altering at least one of the changed color's intensity, shade, hue, saturation, and brightness.

19. The method of claim 14, wherein the at least one catheter further includes a center electrode.

20. The method of claim 14, wherein the at least one catheter includes a treatment catheter and a mapping catheter.

21. The method of claim 14, wherein the baseline impedance value is determined by measuring impedance in blood.

22. A system for delivering therapeutic ablative energy to tissue, the system comprising:
at least one catheter;
an energy delivery body configured with the at least one catheter, wherein the energy delivery body includes a plurality of spline electrodes that are arranged circumferentially to form a basket structure;
a reference electrode located within the basket structure and configured to function as an impedance sensor;
at least one processor configured by executing instructions stored on processor-readable media to process information associated with the impedance sensor; and
a display that is configured to provide information processed by the at least one processor,
wherein the at least one processor is further configured to:
calculate an initial baseline impedance value for each of the plurality of spline electrodes, wherein the baseline impedance value for each spline electrode is based on impedance sensed by the impedance sensor; and
display, on the display, an impedance indicator that indicates, for each spline electrode a change in impedance from the baseline impedance value to a present impedance value measured in real time by the impedance sensor for the respective spline electrode;
wherein the tissue modification apparatus delivers the therapeutic energy via the energy delivery body.

23. The system of claim 22, wherein the reference electrode is located at a distal end of the basket.

24. The system of claim 22, wherein the reference electrode is located at a proximal end of the basket.

25. The system of claim 24, wherein the baseline impedance value and the present impedance value for each spline electrode are based on an impedance calculation between the reference electrode and the respective spline electrode.

26. The system of claim 22, wherein a proximal end region of each spline electrode has insulating material and the reference electrode is disposed distal to the insulating material at a distal end region of the basket, wherein a length of the spline electrode distal to the insulating material comprises an exposed metal electrode.

27. The system of claim 22, wherein the processor is further configured to calculate for each spline electrode a contact stability value and compare it to a threshold contact stability value to determine whether the respective electrode contact with tissue is stable over a predetermined period of time.

28. The system of claim 22, wherein the processor is further configured to provide haptic feedback to a user by generating a vibration or pulse in a handle of the at least one catheter which provides confirmation that at least a threshold degree of contact between the energy delivery body and tissue has been achieved and delivery of the therapeutic energy can begin.

29. The system of claim 22, wherein the processor is configured to determine an integrity of the present impedance value of one respective spline electrode by comparing the present impedance value of one respective spline electrode to the present impedance value of adjacent spline electrodes.

30. The system of claim 29, wherein if the difference in measured impedance values between the one respective spline electrode and each of the adjacent spline electrodes exceeds a predetermined threshold, then the processor flags the impedance value of the one respective spline electrode as being unreliable.
